(19) United States
(12) Patent Application Publication
(10) Pub. No.: US 2006/0024314 A1
(43) Pub. Date: Feb. 2, 2006

(54) Antibodies and cyclic peptides which bind CEA (carcinoembryonic antigens) and their use as cancer therapeutics

(76) Inventors: Clifford P. Stanners, Ontario (CA); Christian Ilantzis, Longueuil (CA); Cosme Ordonez-Garcia, Montreal (CA); Maryam Taheri, Pierrefonds (CA); Robert A. Screaton, La Jolla, CA (US); Abraham Fuks, Montreal (CA); H. Uri Saragovi, Montreal (CA)

Correspondence Address: Ralph A. Dowell of DOWELL & DOWELL P.C., 2111 Eisenhower Ave., Suite 406, Alexandria, VA, 22314, US

(21) Appl. No.: 11/097,224

(22) Filed: Apr. 4, 2005

Related U.S. Application Data
(63) Continuation of application No. PCT/CA03/01533, filed on Oct. 3, 2003.
(63) Continuation-in-part of application No. 11/041,199, filed on Jan. 25, 2005.
Application No. 11/041,199 is a continuation of application No. 09/637,530, filed on Aug. 11, 2000, now abandoned.
Application No. 09/637,530 is a continuation of application No. PCT/CA99/00119, filed on Feb. 11, 1999.
(60) Provisional application No. 60/415,520, filed on Oct. 3, 2002.
(60) Provisional application No. 60/415,520, filed on Oct. 3, 2002.

(30) Foreign Application Priority Data
Feb. 12, 1998 (CA) 2,224,129

(51) Int. Cl. A61K 38/17; A61K 38/12; A61K 39/395

(52) U.S. Cl. 424/155.1; 514/12; 514/9

(57) ABSTRACT

The present invention relates to differentiation and tumorigenicity. The present invention more particularly relates to ligands which target CEA and CEACAM6 such that the adhesion, differentiation-Inhibitory activities and tumorigenic effects of Ig superfamily members, CEA and CEACAM6, can be reduced or blocked. More particularly, the present invention relates to CEA-binding agents which reverse CEA-mediated tumorigenic effects by declustering CEA and CEACAM6. In one embodiment the invention relates to methods of reducing, preventing or reversing a CEA-mediated tumorigenic effect comprising a use of a CEA-mediated tumorigenic effect reducing CEA-declustering agent. In one embodiment, the invention relates to compositions and use thereof for reversing CEA-mediated tumorigenic effects on human cancer cells and uses thereof. In particular, the application relates to a monovalent CEA binding agent which interferes with a CEA interaction responsible for a CEA-mediated tumorigenic effects, thereby minimizing or reversing same.

### CROSS-REFERENCE TO RELATED APPLICATIONS

This application is a continuation application of PCT/CA2003/001533 filed Oct. 3, 2003, which is an international filing claiming priority from U.S. provisional application No. 60/415,520, filed on Oct. 3, 2002. This application is also a continuation-in-part of U.S. patent application Ser. No. 11/041,199, filed Jan. 25, 2005, which is a continuation application of U.S. patent application Ser. No. 09/637,530, filed Aug. 11, 2000 and now abandoned, which is a continuation application of PCT/CA99/00119, filed Feb. 11, 1999, which is an international filing of Canadian Patent Application No. 2,224,129, filed Feb. 12, 1998, all of which are incorporated herein by reference.

### FIELD OF THE INVENTION

The present invention relates to differentiation and tumorigenicity. The present invention more particularly relates to ligands which target CEA such that the adhesion, differentiation-Inhibitory activities and tumorigenic effects of Ig superfamily member, CEA, can be reduced or blocked. More particularly, the present invention relates to CEA-binding agents which reverse the CEA-mediated tumorigenic effects. In one embodiment the invention relates to methods of reducing, preventing or reversing CEA-mediated tumorigenic effects comprising a use of an amount of a CEA declustering agent that can reverse a CEA mediated tumorigenic effect. In one embodiment, the invention relates to compositions for reversing CEA-mediated tumorigenic effects on human cancer cells and uses thereof.

### BACKGROUND OF THE INVENTION

Tumor cells at many sites, including colon, breast, lung, cervix, ovary, stomach, bladder, pancreas and esophagus express large amounts of carcinoembryonic antigen (CEA) and/or the closely related family member, NCA, now termed CEACAM6, on their surfaces. The expression of these glycoproteins, especially CEA, in normal cells is very limited. This represents the basis for the wide clinical use of CEA as a blood tumor marker. Since the majority of human cancers show up-regulation of CEA/CEACAM6, any therapy based on this fact has potential application to an immense number of cancer patients.

Since CEA family members are over-produced in over 50% of all human cancers and are expressed on the surface of the tumor cells in these cancers, they thus represent appealing targets for anti-cancer therapies. We have shown that de-regulated over-expression of CEA and CEACAM6 have tumorigenic effects on human cancer cells in that cell differentiation and polarization are blocked, anoikis is inhibited and tissue architecture is disrupted. The reversal of these effects with a number of different agents could thus be the basis of a novel anti-cancer treatment. One type of agent in the application is the Fab fragment of a mAb directed against certain subdomains in the N domain of CEA necessary for self interaction of the external domains—such self interaction is required for the tumorigenic effects of CEA and CEACAM6. One such mAb is A20.

Tissue architecture is established and maintained to a large extent by specific affinities of cell surface glycoproteins for molecules in the extracellular matrix or on the surface of adjacent cells; the latter are known collectively as cell adhesion molecules (CAMS) (1-3). CAMs function not only to fix cells in specific locations within tissues and regulate their movement but also to translate biochemical information from the extracellular environment through the activation of intracellular signaling pathways leading to specific cell functional responses (4,5). CAMs are grouped into several different molecular families; the majority identified to date belong to the immunoglobulin superfamily (IgSF) (1,6-8). Although the members of this family are functionally diverse, most are cell surface molecules involved in the recognition of other soluble or cell associated molecules. All members of the IgSF share conserved amino acid residues that are limited to positions within the core of the Ig fold important for its structure. In contrast, the functional regions of the various members are often highly diverse.

IgSF members function in many cases as a result of homophilic or heterophilic binding interactions between their external domains (1,9,10). Homophilic interactions can be either anti-parallel or parallel. Anti-parallel interactions between molecules on apposed cell surfaces are required for intercellular adhesion (9,11,12). Parallel interactions between adjacent molecules on the same cell surface can facilitate this process by concentrating the binding molecules into synergistic arrays, as described by the “velcro” (13) or “zipper” models (14), in which the concerted action of multiple relatively weak interactions between individual pairs of molecules can lead to a strong overall bonding. Both types of interactions can also initiate signaling events (5,14,15). The clustering resulting from their combination might be expected to amplify these signals, leading to the triggering of threshold-activated signaling pathways (5,14,15).

The human carcinoembryonic antigen (CEA) family is of particular interest in terms of these considerations because of the multiplicity and diversity of interactions between multiple, closely related family members (13). The structural requirements for CEA-mediated intercellular adhesion has been previously investigated (9,16). Intercellular adhesion seems to depend mainly on anti-parallel CEA-CEA interactions, as indicated by studies with hybrid constructs between CEA and the neural cell adhesion molecule (NCAM) (9). CEA consists of a V-like Ig amino-terminal N domain, followed by three pairs of 1-like Ig domains (AxBx) terminated by a hydrophobic domain that gets processed to allow the addition of a glycophosphatidyl inositol (GPI) membrane anchor (17-19). Intercellular adhesion was shown to be mediated by double reciprocal bonds between the N and A3B3 domains of anti-parallel CEA molecules on apposed cell surfaces (9). Subdomains of 5-6 amino acids that were required for intercellular adhesion, presumably as points of initial binding, were identified by mutational analysis of the N domain (16).

Two cellular functions, intercellular adhesion [for review, see Stanners and Fuks, (13)] and the inhibition of cell differentiation (20) have been shown to be dependent on homophilic CEA family member interactions. In particular, CEA was shown to block the myogenic differentiation of rat L6 myoblasts (20) and the neurogenic differentiation of mouse P19 embryonal carcinoma cells. The effect of CEA on differentiation would be expected to promote tumorigenic behavior and, in fact, was found to markedly increase the tumorigenicity of rat L6 myoblasts (21) and human colorectal Caco2 cells (41). De-regulated over-expression of CEA and closely related CEA family member, CEACAM6 (formerly NCA), at levels closely approximating those found in many colorectal carcinomas, has been shown recently to block cellular polarization, disrupt tissue architecture and block the differentiation of human colonocyte cell lines (41). Consistent with these results, the cell surface level of CEA determined by FACS analysis of highly purified epithelial colonocytes from colorectal carcinomas and normal colonic tissue was found to be elevated in the tumor cells at levels that were inversely correlated with the degree of differentiation (22).

Since CEA and/or CEACAM6 are over-expressed in over 50% of human cancers (24), it would seem experimentally and medically important to devise a means of releasing the CEA-mediated tumorigenic effects. A determination as to whether interference with this function can be made selective without affecting the intercellular adhesion function is also of interest. In any event, there thus remains a need to provide ligands and molecules which are capable of releasing the CEA/CEACAM6-mediated tumorigenic effects. Should it be shown that the interference with the differentiation block can be made selective, it would be desirable to provide such selective molecules.

The present invention seeks to meet these and other needs.

The present description refers to a number of documents, the content of which is herein incorporated by reference in their entirety.

### SUMMARY OF THE INVENTION

The invention concerns monovalent molecules which can interfere with CEA interactions responsible for the CEA-mediated tumorigenic effects.

The present invention further relates to methods which identify molecules that can interfere with CEA interactions responsible for the CEA-mediated tumorigenic properties.

In another embodiment, the present invention provides the means to further improve the potency and/or selectivity of agents that can reverse the CEA-mediated tumorigenic properties.

In yet another embodiment, the invention relates to means to render the binding agents effective at reversing CEA-mediated tumorigenic properties.

In addition, the invention relates to a method of reversing a CEA-mediated tumorigenic property in a cell, a tissue or an animal, comprising the administration to the cell, tissue, or animal, of an amount of a CEA declustering agent which interferes with CEA interactions responsible for the tumorigenic property.

The present invention shows that the agents of the present invention which interfere with CEA interactions minimize CEA-CEA interactions between cells and between molecules on the same cell, and preferably, inhibits or abrogates same.

Prior to the present invention, ligands or agents which would bind to CEA were thought to all have the potential of releasing the differentiation block and reversing the CEA-mediated tumorigenic effect. The present invention shows that divalent ligands, not only do not reverse the CEA-mediated tumorigenic effects, but actually actively promote them.

Thus in accordance with the present invention, the CEA-interaction interfering agent which reverses the CEA-mediated tumorigenic properties is shown to be monovalent.

In one particular embodiment, the agent which interferes with the CEA interaction is a monovalent ligand which cannot promote aggregation between cells and which inhibits and/or reverses crosslinking or clustering of CEA/CEACAM6 molecules.

The terminology “inhibition” or “interference” of “CEA interactions” or the like with a monovalent CEA binding agent include inhibition of parallel interactions, anti-parallel interactions or both.

The present invention therefore relates to monovalent CEA binding agents.

The terminology “monovalent CEA binding agent” will be easily understood by the person of ordinary skill to which the present invention pertains. A non-limiting example of a monovalent CEA-interacting agent includes peptides, more particularly cyclized peptides which are derived from a CEA region implicated in CEA-mediated tumorigenic properties. In particular, the CEA sequence is chosen from one of the three subdomains of the N-terminal CEA region, or a region which bridges these three subdomains and is involved in CEA-CEA interactions. In a particular embodiment, the CEA sequence serves as the template to rationally select and design monovalent agents which interfere with CEA interactions. The present application also relates to small cyclized peptides. In another particular embodiment, the present invention relates to monovalent antibodies and derivatives thereof. Non-limiting examples of such monovalent antibodies include monovalent Fab fragments of monoclonal antibodies which have been designed and that are capable of reversing the tumorigenic properties mediated by CEA interactions, releasing the differentiation block by declustering CEA molecules on the cell surface.

The present application also relates to the showing that, although the structural requirements for the intercellular adhesion and the differentiation block functions overlap, they can be effectively separated in the case of rat L6 myogenic differentiation.

Herein, some work is focused on the effects of CEA on the differentiation of L6 myoblasts because of relative ease of experimentation. Although the expression of CEA in this system is ectopic, experiments to date indicate that results obtained with this model system are closely mimicked by more biologically relevant systems such as human colonocytes (23), 41. Thus, the L6 myoblast system is an accurate cell model system for the studies presented herein. Consequently, not only are the results obtained herein predicted to be translatable to more natural cell systems in which CEA family members expression is encountered, but in fact they are translatable in view of the results obtained with human colonocytes.

In a further aspect, the invention relates to methods for identifying agonists and antagonists using the materials provided by the invention. In a related aspect, the invention relates to methods for treating CEA-associated disorders, and in particular CEA-mediated with the identified agonist or antagonist.

It is believed that prior to the present invention, it was unknown that divalent CEA-binding agents would promote CEA-mediated tumorigenic effects, while monovalent CEA-binding agents would reverse same. The present invention therefore teaches that monovalent agents should be chosen as potential CEA-targeting therapeutic agents.

It should be understood that any monovalent ligand or divalent ligand which can be transformed into a monovalent ligand, wherein such ligands affect the CEA-CEA interaction defined herein are encompassed by the present invention. A number of CEA-specific antibodies and CEA-specific monoclonal antibodies are known in the art. Such known CEA-specific antibodies rendered monovalent could be shown in accordance with an assay of the present invention as exhibiting an anti-CEA-mediated tumorigenic effect. Non-limiting examples of such antibodies encompassed by the present invention include A20, B18, D13, D6, R19, L12 and B9 monoclonal antibodies. In one particular embodiment of the invention, there is thus provided a method of identifying a monovalent compound that is active on a first polypeptide of CEA (or CEACAM6), or a biologically active fragment, or variant thereof, wherein the CEA or CEACAM6 polypeptide or biologically active fragment or variant thereof is capable of binding specifically with a second CEA or CEACAM6 polypeptide, a biologically active fragment thereof or variant thereof, wherein the fragments or variants retain their capability of binding to the first CEA or CEACAM6 polypeptide, fragment, or variant thereof. For certainty, the first and second polypeptides can be CEA or CEACAM6 or a biologically active fragment thereof or variant thereof (homoCEA interactions). Alternatively, the first and second polypeptides can be either of CEA or CEACAM6, or biologically active fragment thereof or variant thereof (heteroCEA interactions). It follows that the designation “CEA”, as used herein also includes CEACAM6, and vice-versa. It should also be understood that the first and second CEA/CEACAM6 polypeptides need not be the same. What is required is that these peptides specifically interact. In a preferred embodiment, the biologically active fragment of CEA or variant thereof is the N-terminal or H region thereof or is derived from the N- or H-region. More particularly, the biologically active fragment or variant of CEA is, or is derived from, the N-terminal region thereof.

In one preferred embodiment of the invention, the identification of a compound active on a CEA polypeptide is provided by a method comprising: contacting a first CEA polypeptide and a second CEA polypeptide with the candidate compound. The first polypeptide comprises an amino acid sequence of CEA, a fragment, or variant thereof that specifically binds to the second CEA polypeptide which can be the same or different from the first CEA polypeptide. A decrease in a detectable biological activity in the presence of the candidate compound (or pool thereof) relative to the biological activity in the absence thereof identifies the candidate compound as a compound that is active on a CEA polypeptide, fragment or variant thereof. In one particular embodiment, the first and second polypeptides, fragment thereof, or variant thereof, are CEA-derived and maintain their biological activity.

In one particular embodiment, the biological activity is the binding of the first and second polypeptides to each other, the method comprising: a) contacting an assay mixture comprising: i) a first CEA polypeptide, or a biologically active fragment, or variant thereof (e.g. the N-terminal region of CEA), and ii) a second CEA polypeptide, a fragment thereof, and a variant thereof; with a test compound; b) measuring the binding of the first and the second polypeptides in the presence of the candidate compound relative to the binding in the absence thereof and; c) determining the ability of the candidate compound to interact with a CEA polypeptide, fragment, or variant thereof, wherein a decrease in the binding of the first and the second polypeptides in the presence of the candidate compound that interacts with the first or second polypeptide, relative to the binding in the absence of the candidate compound, identifies the candidate compound as a compound that is active on a CEA polypeptide, fragment or variant thereof.

In one embodiment such a screening assay mixes the polypeptides of the present invention with a monovalent candidate compound or pool thereof. In another embodiment, the candidate compounds which are selected in a primary screen, are not monovalent but are derivatized into monovalent compounds and used in a second (or third) screening assay (see below).

In one embodiment, the step of detecting comprises the step of measuring the binding of the first and second proteins, wherein the first or the second protein is directly or indirectly labeled with a detectable moiety.

In different embodiments, the step of detecting comprises, but is not limited to, measurement by the method selected from the group consisting of time-resolved fluorescence resonance energy transfer, fluorescence polarization changes, measurement by surface plasmon resonance, a scintillation proximity assay, and a biosensor assay.

In one embodiment, a library of compounds is used. Non-limiting examples of candidate compounds include a small molecule, a peptidomimetic compound, a peptide, and most preferably, antibodies and fragments or derivatives thereof.

The invention also encompasses a method of identifying an antitumorigenic monovalent agent comprising determining whether a test compound is active on a CEA, or parts thereof.

The invention further encompasses a method of identifying a compound that is active on a CEA polypeptide, a fragment or a variant thereof, comprising the steps of contacting a candidate compound (or library thereof) with cells expressing CEA (naturally or not); and detecting CEA activity in the cells, wherein a decrease in activity relative to CEA activity in cells not contacted with a candidate compound is indicative of inhibition of CEA activity. The invention also encompasses such a method but using a fragment or variant of CEA, wherein the fragment or variant retains its biological activity (e.g. in CEA-CEA interaction).

Of course, the invention further encompasses methods of identifying a compound that modulates the activity of a CEA polypeptide, wherein a compound increasing the activity relative to CEA activity in cells not contacted with the candidate compound, is selected as a compound which is a stimulator of CEA activity.

In a preferred embodiment, the step of detecting comprises a method of measuring the ability of a candidate, test compounds, or agents to stimulate or preferably to inhibit a CEA molecule's ability to effect clustering of CEA-expressing cells (such assays are described in more detail hereinbelow).

The invention further encompasses a method of identifying a compound that is active on a CEA polypeptide, a fragment or a variant thereof, comprising the steps of contacting a candidate compound (or library thereof) in a cell-free assay, with a first and second CEA protein or biologically active portion thereof, either naturally occurring or recombinant in origin; and detecting CEA interaction, wherein a decrease in CEA interaction in the cell-free assay when compared to CEA interaction measured in the absence of the candidate compound is indicative of inhibition of CEA activity. In one particular embodiment of this aspect of the present invention, interaction domains of CEA are fused to heterologous polypeptide sequences and the activity of the now joined heterologous polypeptides, through the CEA-CEA interaction, is detected.

As alluded to above, the assays described herein may be used as initial or primary screens to detect promising lead compounds for further development. The same assays may also be used in a secondary screening assay to measure the activity of candidate compounds on a CEA polypeptide. Often, lead compounds will be further assessed in additional, different screens. This invention also includes secondary CEA screens which may involve biological assays in cells.

Tertiary screens may involve the study of the effect of the agent in an animal. Accordingly, it is within the scope of this invention to further use an agent identified as described herein in an appropriate animal model. For example, a test compound identified as described herein (e.g., a monovalent antibody which interferes with CEA-CEA interactions in a cell free assay, and perhaps in a cell-based assay) can be used in an animal model to determine the efficacy, toxicity, or side effects of treatment with such an agent. Furthermore, this invention pertains to uses of novel agents identified by the above-described screening assays for treatment (e.g. cancer), as described herein.

The invention further encompasses a method of making an anticancer compound, comprising the steps of: a) determining whether a candidate compound is active on a CEA polypeptide, fragment or variant thereof, or a nucleotide sequence encoding the polypeptide, fragment or variant thereof; and b) synthesizing or purifying the candidate compound in an amount sufficient to provide a therapeutic effect when administered to an animal affected by CEA-CEA-mediated interactions. The invention encompasses the use of an animal model for tumorigenicity or other CEA-mediated diseases or conditions.

It should be understood that more than one active monovalent agent of the present invention could be combined.

The invention further encompasses a method for treating or preventing a CEA-mediated tumorigenic effect in an animal suffering from, or susceptible of suffering from same, comprising administering thereto a therapeutically effective amount of a monovalent CEA-binding agent, or nucleic acid sequence encoding same. The animal is preferably a primate, and more preferably a human.

The invention further encompasses a method of prophylactic treatment to prevent a CEA-mediated tumorigenic effect in an animal at risk of developing a CEA-mediated cancer comprising administering thereto a prophylactically effective amount of a monovalent CEA-binding agent, or nucleic acid sequence encoding same in an amount sufficient to prevent CEA-mediated cancer of the animal. In a particular embodiment, the prophylactically effective amount reduces CEA-dependent adhesion of cells. In a particular embodiment, the prophylactically effective amount reduces or abrogates CEA-mediated clustering of cells in a mammal.

The invention further encompasses an isolated, purified or enriched monovalent antibody, fragment or derivative thereof which is specific for a CEA region involved in CEA-CEA interaction.

The invention further encompasses a composition comprising two CEA polypeptides which specifically interact, and a monovalent agent which interferes with the CEA-CEA interaction. In a particular embodiment, the monovalent agent which interferes with the CEA-CEA interaction, is a monovalent antibody, fragment or derivative thereof.

Further, the invention encompasses a process for producing a pharmaceutical composition comprising: a) carrying out a screening assay of the present invention aimed at identifying a monovalent agent that interferes with a CEA-CEA interaction, wherein a first CEA polypeptide is capable of binding specifically with a second CEA polypeptide (or fragment or derivative thereof, which maintains the capability of specific interaction), and wherein the screening assay enables the identification of a candidate agent as an agent that interferes with the CEA-CEA interaction; and b) mixing the compound identified in a) with a suitable pharmaceutical carrier. In one embodiment, the agent is monovalent. In another embodiment the agent which is selected as an agent which interferes with the CEA-CEA interaction, is not monovalent, and the process further comprises a step of modifying the non-monovalent agent to obtain a monovalent CEA-CEA interaction interfering agent prior to step b). In one embodiment, the CEA polypeptides are chosen from full-length CEA (or CEACAM6, see above) polypeptides, genetically engineered CEA polypeptides (designed to reflect the sequence of mature CEA), or fragments or derivatives of CEA polypeptides, which retain their biological activity of specifically interacting together.

In a further embodiment of this process of producing a pharmaceutical composition, the process further includes a scaling-up of the preparation for isolating of the identified and selected CEA interaction interfering monovalent agent. In yet another embodiment of this process of producing a pharmaceutical composition, the pharmaceutical composition prepared comprises a derivative or homolog of the compound identified in a), above.

Also, the invention encompasses the use of a pair of CEA polypeptides, biologically active fragment thereof or variants thereof, wherein the CEA polypeptides are capable of binding specifically to each other; for the identification of an agent which interferes with the specific CEA interaction of CEA polypeptides, biologically active fragments thereof, or variants thereof.

### BRIEF DESCRIPTION OF THE DRAWINGS

Having thus generally described the invention, reference will now be made to the accompanying drawings, showing by way of illustration a preferred embodiment thereof, and in which:

FIG. 1 shows fusion indices after cross-linking of ΔNCEA molecules in differentiation-competent L6 (ΔNCEA) transfectants with polyclonal and monoclonal anti-CEA antibodies. Purified rabbit polyclonal and mAb D14, recognized intact epitopes in ΔNCEA and converted ΔNCEA to a differentiation-blocking molecule, whereas control antibodies A20 and B18, directed to epitopes not present in ΔNCEA, had no effect. Values shown represent the mean and standard error of measurements from 2 independent experiments.

FIG. 2 shows a schematic diagram of the structure of CEA showing the major domains and subdomains in the N domain selected for intensive study and the amino acid sequence of the N-terminal domain of CEA.

FIG. 3 shows FACS distributions relating the number of cells with the level of cell surface expression of wild type and mutant CEA in L6 myoblast transfectant and control (Neo) populations. All cells were labeled with monoclonal anti-CEA antibody, J22. Immunoreactivity was detected with goat anti-mouse FITC-conjugated antibody.

FIG. 4 shows photomicrographs and fusion indices of L6 Neo, L6(CEA), and L6 subdomain 1 mutant CEA transfectants subjected to the myogenic differentiation assay.

FIG. 5 shows photomicrographs and fusion indices of L6 Neo, L6(CEA), and L6 subdomain 2 mutant CEA transfectants subjected to myogenic differentiation assay.

FIG. 6 shows photomicrographs and fusion indices of L6 Neo, L6(CEA), and L6 subdomain 3 mutant CEA transfectants subjected to myogenic differentiation assay.

FIG. 7 shows the effect of amino acid substitutions at position Q80 and D82 on the kinetics of CEA-mediated cell-cell aggregation of corresponding LR-73 cell transfectants. LR(Neo)—negative control, LR(CEA)—positive control. FACS profiles indicating expression levels of CEA and mutant CEA in these transfectants utilizing mAb J22 are given in (16). Mean levels of expression in arbitrary units were LR(CEA), 420; LR(Q80R), 388; and LR(D82N), 249.

FIG. 8 shows immunoblot results for SDS PAGE gels of extracts of LR-73 cells stably transfected with the indicated CEA mutants in subdomains 1 and 2. CEA mutant protein was detected with mAbs A20, B18 and J22 with qualitative results indicated in A and gel patterns for B18 shown in B. Primary gel data for A20 is shown in Taheri et al. (16).

FIG. 9 shows photomicrographs of L6 (Neo), L6 (CEA) and L6(CEA) cultures treated with cyclized peptides at a concentration of 100 μm representing subdomains 1, 2, and 3 and with monovalent Fab fragments of mAbs A20, B18 and J22 at a concentration of 100 μg/ml, after growth in DM. Differentiation is indicated by immunofluorescence with anti-myosin antibody. The background fluorescence seen in the L6(CEA) culture in the mAb series was due to loss of CEA expression by occasional cells in the cultures used for this series of experiments. Values for fusion index represent averages of 3 independent experiments.

FIG. 10 shows formation of glandular spheroids with polarized cells after treatment of human colorectal carcinoma (A) SW-1222 cells with Mab A-20 Fab preparations, A-20 whole antibody and cross-linked A-20 whole antibody. (B) Formation of glandular spheroids with polarized cells after treatment of human colorectal carcinoma LS-180 cells with Fab preparations of Mab A-20.

FIG. 11 shows phase contrast micrographs of LS-180 spheroids colonies showing spherical well-formed glandular structures after treatment with A-20 Fabs (top panel) and irregular and poorly formed colonies after treatment with control Fabs (bottom panel).

FIG. 12 shows tumor formation (spheroid growth) of LS-174T human colon cancer cells in collagen gel is dramatically inhibited (right) by anti-CEA Fab fragments but not with control Fabs (left).

FIG. 13 shows that CEA/CEACAM6 colocalizes with integrin α5β1. Results of confocal microscopy of L6 cells transfected with CEA, or with CEACAM6 are shown. CEA family members, are stained green with FITC-coupled anti-mouse secondary antibodies whereas integrin α5 is stained red with Rhodamine-coupled anti-hamster secondary antibodies. Merged images in the bottom panel show extensive colocalization of CEA and CEACAM6 with α5β1 integrin (e.g. arrows). Note that colocalization is nearly complete, but not 100% (arrowheads).

FIG. 14 shows that antibody-crosslinking of ΔNCEA molecules recruits integrin-linked kinase (ILK) into lipid rafts. Localization of ΔNCEA and ILK before and after clustering of ΔNCEA by treating L6 ΔNCEA transfectant cells with anti-CEA mAb J22 followed by an anti-mouse IgG secondary Ab is shown. Both antibody treatments were applied for 5 min. at 37° C. Cells were extracted with mild detergent and the extracts subjected to isopycnic sucrose density gradient fractionation by ultracentrifugation. Lipid cell membrane rafts are found in the less dense fractions #3 to #7. Clustering of ΔNCEA rapidly moves ILK into CEA and α5β1 integrin (not shown)-containing membrane rafts.

FIG. 15 shows that treatment with a composition comprising A-20 Fab fragment reverses the colon cancer tumor size in vivo. Hematoxylin stained sections of mini-colons showing a much larger and more solid tumor-like growth of human colon cancer SW-1222 cells (3%) in mixed aggregates with normal rat cells (tissue above the dotted line with kidney below) after control treatment (left panel) compared to treatment with an A-20 Fabs containing composition (right panel).

FIG. 16 shows that A-20 Fab composition treatment increases the differentiation level in vivo in mini-colons. Immunostained sections of A-20 versus control mini-colons. CEA positive cells in A-20 treated cases (right panel) show good glandular architecture; cells are well polarized with apically expressed CEA and basally-oriented nuclei (black arrows). CEA positive cells in control mini-colons (left panel) are poorly differentiated with CEA detected over their entire surfaces and form amorphous collectives lacking tissue architecture and polarized cells (white arrows).

FIG. 17 shows the timing of α5β1 activation as indicated by binding of fibronectin to ΔNCEA transfectants of L6 cells vs time after antibody-mediated cross-linking of ΔNCEA molecules. Two peaks of activation were reproducibly observed at 2 and 30 min.

FIG. 18 shows an alignment of the amino acid sequences of CEA and NCA (now termed CEACAM6) which show their very high homology and enables a predicting of sequences and domains which could be used in accordance with the present invention, as well as to identify amino acids which are important, crucial or essential for CEA-CEA interaction.

FIG. 19 shows the nucleic acid sequences of human CEA cDNA showing for example the structure of the mature protein, and the signal sequence.

FIG. 20 shows the nucleic acid sequences of human CEA CAM6 cDNA showing for example the structure of the mature protein, and the signal sequence.

Other objects, advantages and features of the present invention will become more apparent upon reading of the following non-restrictive description of preferred embodiments with reference to the accompanying drawings which are exemplary and should not be interpreted as limiting the scope of the present invention

### DESCRIPTION OF THE PREFERRED EMBODIMENTS

In order to provide a clear and consistent understanding of terms used in the present description, a number of further definitions are provided herein below.

The terminology “CEA” as used herein is meant to cover CEA and/or CEACAM6 (CEA/CEACAM6.

The terminology “monovalent binding agent”, “monovalent agent”, “monovalent molecule” or the like refers broadly to monovalent agents which interfere with CEA interactions thereby inhibiting same. More particularly these monovalent agents interfere with the clustering of CEA molecules. In one embodiment, the monovalent agent could be selective in that it only interferes with the tumorigenic properties and not with intercellular adhesion. Non-limiting examples of monovalent binding agents include monovalent ligands which bind to a CEA region involved in CEA-CEA interaction (such agents are also broadly referred to as declustering agents); Fab fragments of antibodies; peptides; and cyclized peptides and other monovalent fragments like scFv, Fab′, Fv, dsFv and the like. Of course, humanized or other genetically engineered monovalent fragments are also encompassed by the present invention.

While the instant invention is mostly exemplified with Fab fragments of mAb A20, the present invention is not so limited. Indeed, the present invention also covers monovalent recombinant forms of antibodies which specifically interact with a CEA epitope and reverse and/or release the CEA-induced effects linked directly or indirectly to such an epitope or epitopes. Such monovalent recombinant antibodies include single chain (scFv) or Fab form thereof. As known in the art, the circulation time and bioavailability of antibodies can be increased by conjugation therewith with polyethelene glycol (PEGylation) in order to increase the biological half-life and reduce immunogenicity in a first generation approach. This could be followed by humanization and PEGylation in a second generation approach. Various PEGylation chemistries and attachment strategies including controlled random PEGylation and/or site directed PEGylation with second generation PEGylation chemistry, can be employed as commonly known in the art. Other means of increasing the half life in vivo are also known in the art, for example, protein fusion to blood borne proteins, such as albumen or transferrin can also be utilized.

The monovalent agents of the present invention or CEA “declustering agents” include for example monovalent antibody fragments in natural or recombinant form (Fab/scFv), cyclized peptides and/or peptide mimetics (small molecule class of declustering agents). These small molecules can be linked to one another or assembled into heteromeric oligomers (with PEG) as long as they do not elicit/cause or stabilize CEA/CEACAM6 clustering which would either have no effect or worse, favor undesirable pro-tumorigenic effects (as exemplified, using divalent monoclonal antibodies). The small molecule class of agents can also be PEGylated or fused to other proteins for improvement of pharmacokinetic properties.

Additional agents could also include the assembly of antibody binding determinants onto a scaffold protein in a manner that preserves binding to antigen (a CEA epitope), as well as the concept of shankless anchors (disclosed in WO 99/41370) which could also be thought of as a different class of declustering agents. That shankless anchors can decluster CEA/CEACAM6 molecules is validated by experiments that show a reduction in the amount of CEA that can be chemically cross-linked (indicating a clustered state) when co-expressed with a mutated CEA molecule that mimics a shankless anchor (data not shown).l

The terminology “active on” refers to a measurable effect of an agent on the CEA polypeptide it is active on (as compared to the activity of the CEA polypeptide in the absence of the agent). The activity referred thereto is any one of a biological activity of one of the polypeptides of the present invention.

As used herein, the terms “inhibit”, “inhibition”, “inhibitory”, and “inhibitor” all refer to a function of reducing a biological activity or function. Such reduction in activity or function can, for example, be in connection with a CEA-mediated function. Non-limiting examples of CEA-mediated effects or function include tissue architecture, differentiation, cell adhesion, tumorigenic effects, and fibronectin binding. Assays to assess these functions are thus encompassed by the present invention (e.g. adhesion assay, spheroid assay, tumorigenic assay).

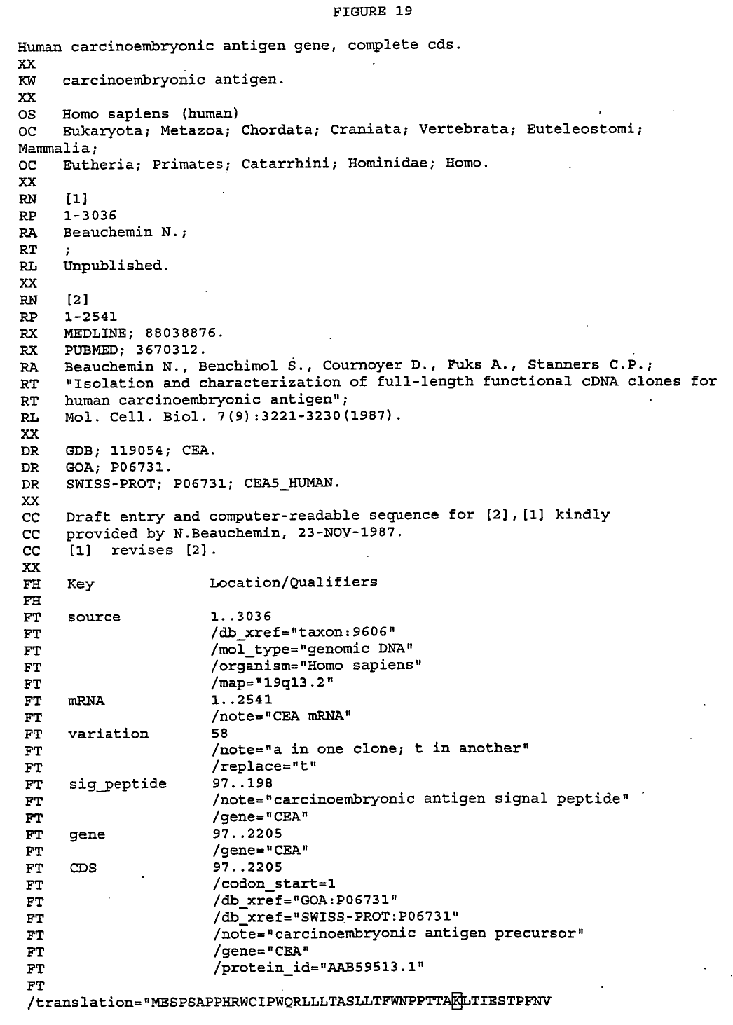

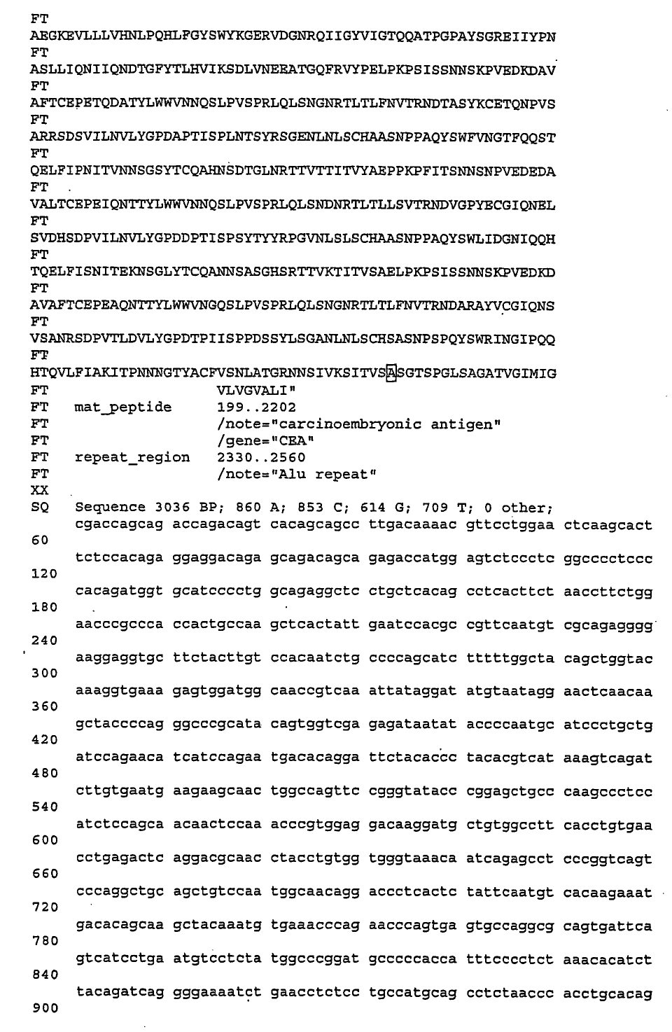

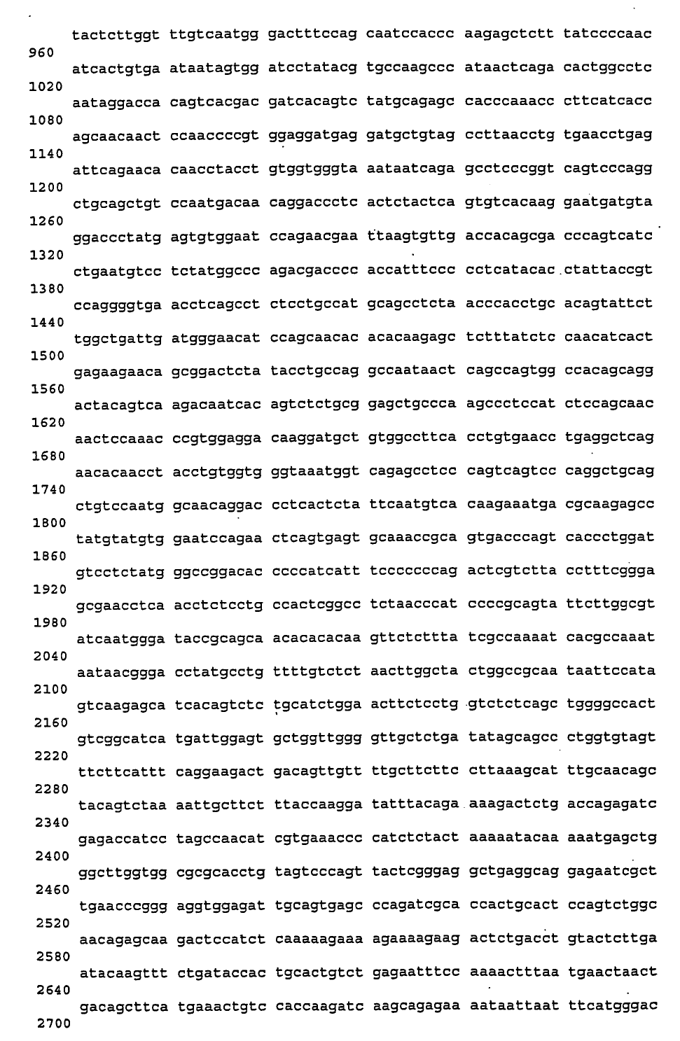

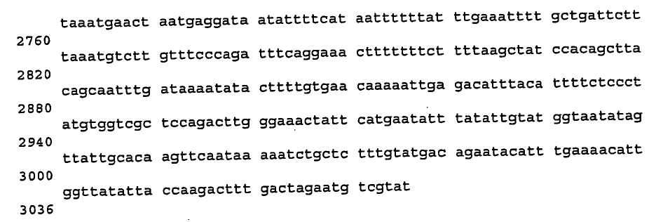

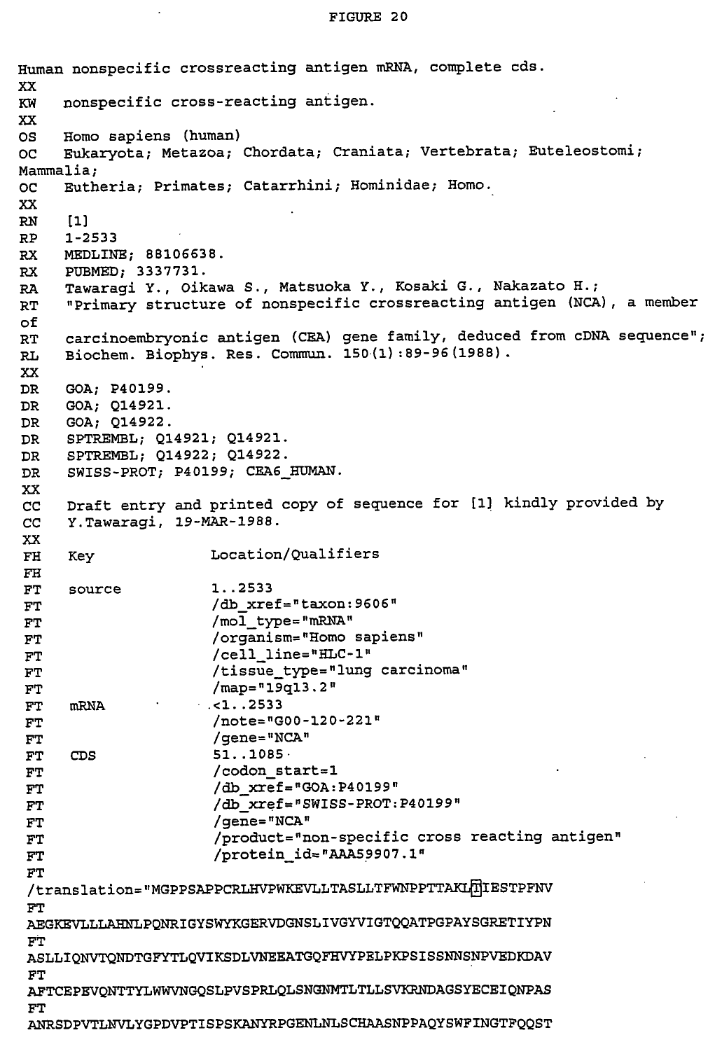

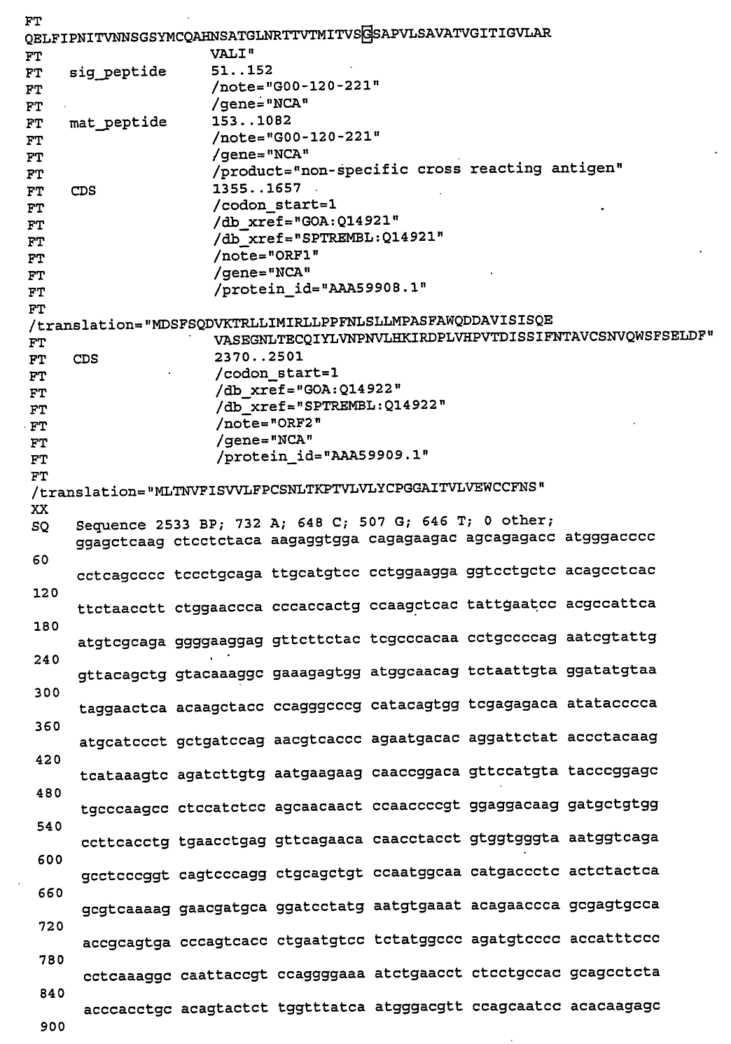

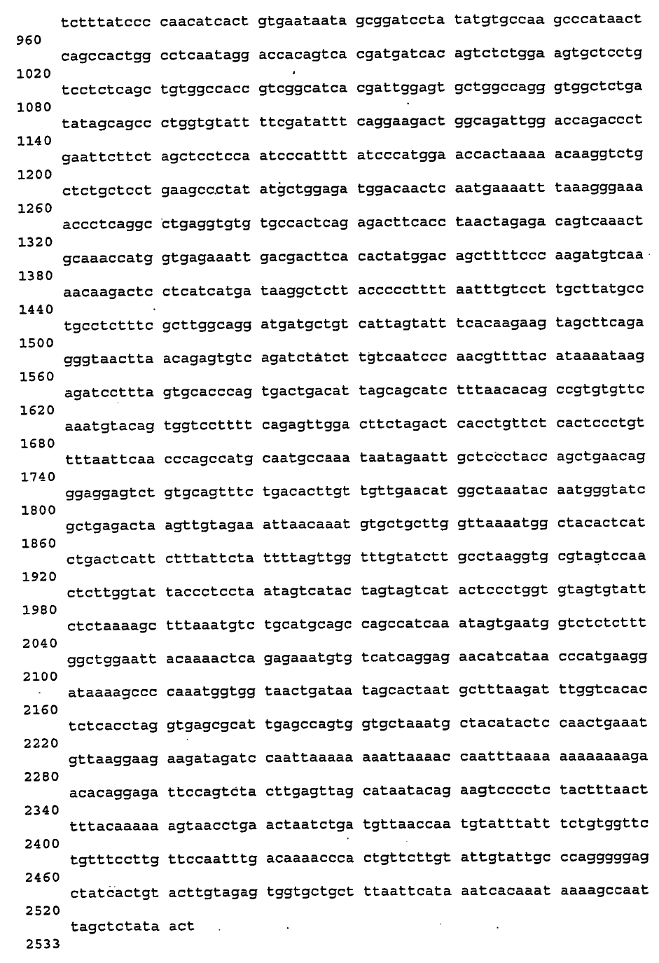

As used herein, the terminology “CEA polypeptide” or the like refers to a CEA or CEACAM6 polypeptide, or a chimera thereof, encompassing for example the CEA or CEACAM6 sequences described in the Figures or in the sequence listing, as well as a variant or derivative thereof. Non-limiting examples of CEA polypeptides include polypeptides comprising the amino acid sequence as set forth in SEQ ID NO: 2, or SEQ ID NO: 4, variants or fragments thereof. Such variants and derivatives thereof include for example a mature form of a CEA polypeptide lacking the leader peptide and/or C-terminal domain thereof (FIGS. 19 and 20).

As used herein, the term “active domain of CEA”, “biologically active polypeptide of CEA” or the like refers to a polypeptide fragment or portion thereof that retains an activity of CEA. The term “CEA polypeptide” is also meant to encompass CEA or an active domain thereof that is fused to another polypeptide, such as a non-CEA polypeptide sequence.

These include, but are not limited to, nucleotide sequences comprising all or portions of the CEA nucleic acids depicted in SEQ ID NOs:1 and 3 which are altered by the substitution, deletion or mutation of different codons that encode a functionally equivalent amino acid residue within the sequence.

“CEA activity”, “polypeptide comprising the amino acid sequence SEQ ID NO: 2 activity”, “polypeptide comprising the amino acid sequence SEQ ID NO: 4 activity”, “CEACAM6 activity” or “biological activity” of CEA or other polypeptides of the present invention is defined as a detectable biological activity of a CEA gene; nucleic acid sequence, protein or polypeptide of the present invention. This includes any physiological function attributable to the specific biological activity of CEA, or CEACAM6 of the present invention. Non-limiting examples include measurement of cell adhesion in vitro (28), of differentiation (e.g. myogenic differentiation block), of fibronectin binding, etc. Non-limiting examples of the biological activities may be made directly or indirectly. Biological activities may also include simple CEA-CEA binding assays. Thus, CEA biological activity includes any standard biochemical measurement of CEA such as conformational changes, phosphorylation status or any other feature of the protein that can be measured with techniques known in the art. CEA biological activity also includes in vivo activities such as the CEA-mediated tumorigenic effect which can be assayed, for example using nude mice as exemplified herein. Furthermore, for certainty, the terminology “biological activity” also includes measurements based on the interaction of interacting domains of CEA proteins or polypeptides of the present invention.

Determining the binding between polypeptides of the present invention can be accomplished by one of the methods described below or known in the art for determining direct binding. While it might be advantageous in certain embodiments of the present invention to provide a binding assay which is amenable to automation and more particularly to high-throughput, the present invention is not so limited. The binding or physical interaction between a CEA polypeptide of the present invention, or fragment thereof (e.g. the N-terminal domain of CEA) may be between isolated polypeptides consisting essentially of the sequence necessary for binding, or, alternatively, the respective polypeptide sequence may be comprised within a larger polypeptide. Of course, CEA polypeptides of intermediate size, as compared to the full length sequences, are also encompassed by the present invention.

A number of non-limiting methods, useful in the invention, to measure the binding of CEA polypeptides are described below. Binding can be measured by coupling one molecule to a surface or support such as a membrane, a microtiter plate well, or a microarray chip, and monitoring binding of a second molecule by any number of means including but not limited to optical spectroscopy, fluorometry, and radioactive label detection.

For example, Time-Resolved Fluorescence Resonance Energy Transfer (TR-FRET), in which the close proximity of two fluorophores, whether intrinsic to, as in the case of a naturally-fluorescent amino acid residue such as tryptophan, or either covalently or non-covalently bound to a separate molecule, causes the emission spectrum of one fluorophore to overlap with the excitation spectrum of the second, and thus dual fluorescence following excitation of only one fluorophore is indicative of binding. An additional assay useful in the present invention is fluorescence polarization, in which the quantifiable polarization value for a given fluorescently-tagged molecule is altered upon binding to a second molecule. Surface plasmon resonance assays can be used as a quantitative method to measure binding between two molecules by the change in mass near an immobilized sensor caused by the binding of one protein from the aqueous phase to a second immobilized on the sensor. A scintillation proximity assay can also be used to measure binding of a pair of CEA polypeptides, or fragment thereof, in which binding in the proximity to a scintillant converts radioactive particles into a photon signal that is detected by a scintillation counter or other detector. Additionally, binding can be evaluated by a Bio Sensor assay, which is based on the ability of the sensor to register changes in admittance induced by ion-channel modulation following binding. Phage display is also a powerful quantitative assay to measure protein:protein interaction using colourimetric ELISA (enzyme-linked immunosorbent assay).

As used herein, the term “polynucleotide encoding a polypeptide” or equivalent language encompasses polynucleotides that include a sequence encoding a polypeptide of the invention, particularly a CEA polypeptide and more particularly a CEA involved in CEA-CEA interaction.

As used herein, the term “polynucleotide(s)” generally refers to any polyribonucleotide or poly-deoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s). Polynucleotides can also be DNA and RNA chimeras.

As used herein, the term “polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene-encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation, selenoylation, sulfation and transfer-RNA mediated addition of amino acids to proteins, such as arginylation, and ubiquitination. See, for instance: Proteins—Structure and Molecular Properties, 2nd Ed., T. E. Creighton, W. H. Freeman and Company, New York (1993); Wold, F., Posttranslational Protein Modifications: Perspectives and Prospects, pgs. 1-12 in Posttranslational Covalent Modification of Proteins, B. C. Johnson, Ed., Academic Press, New York (1983); Seifter et al., Meth. Enzymol. 182:626-646 (1990); and Rattan et al., Protein Synthesis: Posttranslational Modifications and Aging, Ann. N.Y. Acad. Sci. 663: 48-62(1992). Polypeptides may be branched or cyclic, with or without branching. Cyclic, branched and branched circular polypeptides may result from post-translational natural processes and may be made by entirely synthetic methods, as well.

As used herein, the term “variant(s)” refers to a polynucleotide or polypeptide that differs from a reference polynucleotide or polypeptide, respectively, but retains one or more of the biological activities of the initial (e.g. non-variant) polynucleotide or polypeptide of the present invention (e.g. CEA). A typical variant of a polynucleotide differs in nucleotide sequence from another reference polynucleotide. Changes in the nucleotide sequence of the variant may or may not alter the amino acid sequence of a polypeptide encoded by the reference polynucleotide. Nucleotide changes may result in amino acid substitutions, additions, deletions, and truncations in the polypeptide encoded by the reference sequence, or in the formation of fusion proteins, as discussed below. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes variants of each of the polypeptides of the invention, that is polypeptides that vary from the referents by conservative amino acid substitutions whereby a residue is substituted by another with like characteristics. Typically, such substitutions are among Val, Leu and Ile; among Ser and Thr; among the acidic residues Asp and Glu; and among the basic residues Lys and Arg; or aromatic residues Phe and Tyr. Particularly preferred are variants in which 1-10, 1-5,1-3, 2-3, or 1 amino acid or amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans. In one embodiment of the present invention, a variant of CEA is thus meant to refer to a sequence thereof which diverges in the sequence of CEA or CEACAM6 which are not involved in CEA-CEA interactions.

As used herein, the term “fragment”, when used in reference to a polypeptide, is a polypeptide having an amino acid sequence that is entirely the same as part but not all of the amino acid sequence of the polypeptide according to the invention from which it “derives”. As with CEA polypeptides, fragments may be “free-standing” (“consisting of”), or comprised within a larger polypeptide of which they form a part or region, most preferably as a single continuous region in a single larger polypeptide.

The term “isolated”, when used in reference to a nucleic acid means that a naturally occurring sequence has been removed from its normal cellular (e.g., chromosomal) environment or is synthesized in a non-natural environment (e.g., artificially synthesized). Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

The term “enriched”, when used in reference to a polynucleotide means that the specific DNA or RNA sequence constitutes a significantly higher fraction (2-5 fold) of the total DNA or RNA present in the cells or solution of interest than in normal or diseased cells or in cells from which the sequence was originally taken. This could be caused by a person, by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased.

As used herein, the term “significantly higher fraction” indicates that the level of enrichment is useful to the person making such an enrichment and indicates an increase in enrichment relative to other nucleic acids of at least about 2-fold, or 5- to 10-fold or even more. The term also does not imply that there is no DNA or RNA from other sources. The other source of DNA may, for example, comprise DNA from a yeast or bacterial genome, or a cloning vector such as pUC19, or eukaryotic cloning vectors. This term distinguishes from naturally occurring events, such as viral infection, or tumor type growths, in which the level of one mRNA may be naturally increased relative to other species of mRNA. That is, the term is meant to cover only those situations in which a person has intervened to elevate the proportion of the desired nucleic acid.

As used herein, the term “purified” in reference to nucleic acid does not require absolute purity (such as a homogeneous preparation). Instead, it represents an indication that the sequence is relatively more pure than in the natural environment (compared to the natural level, this level should be at least 2-5 fold greater, e.g., in terms of mg/mL). Individual clones isolated from a genomic or cDNA library may be purified to electrophoretic homogeneity. The claimed DNA molecules obtained from these clones could be obtained directly from total DNA or from total RNA. cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message over its proportion in naturally occurring cells. Thus, purification of at least one order of magnitude, preferably two or three orders, and more preferably four or five orders of magnitude is expressly contemplated. A genomic library can be used in the same way and yields the same approximate levels of purification.

The terms “isolated”, “enriched”, and “purified” used with respect to nucleic acids, above, may similarly be used to denote the relative purity and abundance of polypeptides. These, too, may be stored in, grown in, screened in, and selected from libraries using biochemical techniques familiar in the art. Such polypeptides may be natural, synthetic or chimeric and may be extracted using any of a variety of methods, such as antibody immunoprecipitation, other “tagging” techniques, conventional chromatography and/or electrophoretic methods. Some of the above utilize the corresponding nucleic acid sequence.

“Identity” and “similarity,” as used herein and as known in the art, are relationships between two or more polypeptide sequences or two or more polynucleotide sequences, as the case may be, as determined by comparing the sequences.

Amino acid or nucleotide sequence “identity” and “similarity” are determined from an optimal global alignment between the two sequences being compared. A non-limiting example of optimal global alignment can be carried-out using the Needleman—Wunsch algorithm (Needleman and Wunsch, 1970, J. Mol. Biol. 48:443-453). “Identity” means that an amino acid or nucleotide at a particular position in a first polypeptide or polynucleotide is identical to a corresponding amino acid or nucleotide in a second polypeptide or polynucleotide that is in an optimal global alignment with the first polypeptide or polynucleotide. In contrast to identity, “similarity” encompasses amino acids that are conservative substitutions.

The term “conservative” substitution is well-known in the art and broadly refers to a substitution which does not significantly change the chemico-physical properties of the substituted amino acid. For example, a “conservative” substitution is any substitution that has a positive score in the blosum62 substitution matrix (Hentikoff and Hentikoff, 1992, Proc. Natl. Acad. Sci. USA 89: 10915-10919). By the statement “sequence A is n % similar to sequence B” is meant that n % of the positions of an optimal global alignment between sequences A and B consists of conservative substitutions. By the statement “sequence A is n % identical to sequence B” is meant that n % of the positions of an optimal global alignment between sequences A and B consists of identical residues or nucleotides. Optimal global alignments can use a number of alignment algorithms (e.g. that of Needleman-Wunsch).

The term ‘identity’ and ‘similarity’ between sequences can be extended to their fragments. An optimal local alignment between sequences A and B is the highest scoring alignment of fragments of A and B. By the statement “sequence A is n % identical locally to B” is meant that n % of the positions of an optimal local alignment between sequences A and B consists of conservative substitutions. By the statement “sequence A is n % similar locally to B” is meant that n % of the position of an optimal local alignment between sequences A and B consists of identical residues or nucleotides. An non-limiting example of optimal local alignment can be carried-out using the Smith-Waterman algorithm [Smith, T. F and Waterman, M. S. 1981. Identification of common molecular subsequences. J. Mol. Biol. 147:195-197].

Of course, the above-listed parameters are but one specific example of alignment algorithm parameters. Numerous algorithms and parameters are available and known to the person of ordinary skill.

Typical conservative substitutions are among Met, Val, Leu and Ile; among Ser and Thr; among the residues Asp, Glu and Asn; among the residues Gin, Lys and Arg; or aromatic residues Phe and Tyr. In calculating the degree (most often as a percentage) of similarity between two polypeptide sequences, one considers the number of positions at which identity or similarity is observed between corresponding amino acid residues in the two polypeptide sequences in relation to the entire lengths of the two molecules being compared.

As used herein, the term “antibody” is meant to encompass constructions using the binding (variable) region of such an antibody, and other antibody modifications. Thus, an antibody useful in the invention may comprise a whole antibody, an antibody fragment, a polyfunctional antibody aggregate, or in general a substance comprising one or more specific binding sites from an antibody. In accordance with the present invention, non-monovalent antibodies will be engineered or modified in order to render them monovalent prior to a screen of the present invention (e.g. a second or third screening assay). The antibody fragment may be a fragment such as an Fv, Fab or F(ab′)₂ fragment or a derivative thereof, such as a single chain Fv fragment. The antibody or antibody fragment may be non-recombinant, recombinant or humanized. The antibody may be of an immunoglobulin isotype, e.g., IgG, IgM, and so forth. In addition, an aggregate, polymer, derivative and conjugate of an immunoglobulin or a fragment thereof can be used where appropriate. Neutralizing antibodies are especially useful according to the invention for diagnostics, therapeutics and methods of drug screening and drug design.

As used herein, the term “specific for an epitope present on a CEA polypeptide”, when used in reference to an antibody, means that the antibody recognizes and binds an antigenic determinant present on a CEA polypeptide or fragment thereof according to the invention.

As used herein, the term “antigenically equivalent derivative(s)” encompasses a polypeptide, polynucleotide, or the equivalent of either which will be specifically recognized by certain antibodies which, when raised to the protein, polypeptide or polynucleotide according to the invention, interferes with the immediate physical interaction between CEA molecules or fragments thereof. As stated, most preferably these equivalents are monovalent.

As used herein, the term “increase in activity” refers to an enhanced level of measurable activity of a polypeptide in a given assay in the presence of a candidate compound relative to the measurable level of activity in the absence of a candidate compound. Activity is considered increased according to the invention if it is at least 10% greater, 20% greater, 50% greater, 75% greater, 100% greater or more, up to 2-fold, 5-fold, 10-fold, 20-fold, 50-fold, 100-fold or more than in the absence of a candidate compound.

As used herein, the term “decrease in activity” refers to a reduced level of measurable activity of a polypeptide in a given assay in the presence of a candidate compound relative to the measurable level of activity in the absence of a candidate compound. Activity is considered decreased according to the invention if it is at least 10% less, preferably 15% less, 20% less, 50% less, 75% less, or even 100% less (i.e., no activity) than that observed in the absence of a candidate compound.

As used herein, the term “conditions that permit their interaction”, when used in reference to a pair of CEA polypeptides, or fragments thereof, and a candidate agent means that the pair and agent are placed together, whether both in solution or with one immobilized or restricted in some way and the other in solution, wherein the parameters (e.g., salt, detergent, protein or candidate compound concentration, temperature, and redox potential, among others) of the solution are such that the pair of CEA polypeptides, and the candidate agent may physically associate. Conditions that permit protein:candidate interaction include, for example, the conditions described herein for TR-FRET, fluorescent polarization, Surface Plasmon Resonance and Phage display assays.

As used herein, the term “detectable change in a measurable parameter of CEA” refers to an alteration in a quantifiable characteristic of a CEA polypeptide.

As used herein, the term “measuring the binding of a candidate compound” refers to the use of an assay permitting the quantitation of the amount of a candidate compound physically associated with a CEA polypeptide, fragment or variant thereof.

A “candidate compound” as used herein, is any compound with a potential to modulate the CEA-CEA interaction.

As used herein, the term “directly or indirectly detectably labeled” refers to the attachment of a moiety to a candidate compound that renders the candidate compound either directly detectable (e.g., an isotope or a fluorophore) or indirectly detectable (e.g., an enzyme activity, allowing detection in the presence of an appropriate substrate, or a specific antigen or other marker allowing detection by addition of an antibody or other specific indicator).

A “method of screening” refers to a method for evaluating a relevant activity or property of a large plurality of compounds, rather than just one or a few compounds. For example, a method of screening can be used to conveniently test at least 100, more preferably at least 1000, still more preferably at least 10,000, and most preferably at least 100,000 different compounds, or even more. In a particular embodiment, the method is amenable to automated, cost-effective high throughput screening on libraries of compounds for lead development.

In a related aspect or in preferred embodiments, the invention provides a method of screening for potential anticancer agents by determining whether any of a plurality of compounds, preferably a plurality of small molecules, is active on CEA. Preferred embodiments include those described for the above aspect, including embodiments which involve determining whether one or more test compounds bind to or reduce the level of activity of a CEA, and preferably decrease or abrogate CEA-CEA interactions involved in CEA-mediated tumorigenic effects.

As used herein, the term “library” refers to a collection of 100 compounds, preferably of 1000, still more preferably 5000, still more preferably 10,000 or more, and most preferably of 50,000 or more compounds.

As used herein, the term “small molecule” refers to compounds having molecular mass of less than 3000 Daltons, preferably less than 2000 or 1500, still more preferably less than 1000, and most preferably less than 600 Daltons. Preferably but not necessarily, a small molecule is not an oligopeptide.

As used herein, the term “mimetic” refers to a compound that can be natural, synthetic, or chimeric and is structurally and functionally related to a reference compound. In terms of the present invention, a “peptidomimetic,” for example, is a non-peptide compound that mimics the activity-related aspects of the 3-dimensional structure of a peptide or polypeptide, for example a compound that mimics the structure of a peptide or active portion of a CEA-CEA interaction domain.

In more than one embodiment of the above assay methods of the present invention, it may be desirable to immobilize one of the interacting CEA polypeptides to facilitate separation of complexed from uncomplexed forms of one or both of the proteins or polypeptides, as well as to accommodate automation of the assay. Binding of a test agent to a CEA protein (or fragment, or variant thereof) or interaction of a pair of CEA proteins in the presence and absence of a candidate compound, can be accomplished in any vessel suitable for containing the reactants. Examples of such vessels include microtitre plates, test tubes and micro-centrifuge tubes.

In one embodiment a fusion protein can be provided which adds a domain that allows one or both of the proteins to be bound to a matrix. For example, glutathione-S-transferase/CEA fusion proteins or glutathione-S-transferase/CEACAM6 can be adsorbed onto glutathione sepharose beads (Sigma Chemical, St. Louis, Mo.) or glutathione derivatized microtitre plates, which are then combined with the test agent and the mixture incubated under conditions conducive to CEA-CEA complex formation (e.g. at physiological conditions for salt and pH). Following incubation the beads or microtitre plate wells are washed to remove any unbound components, the matrix immobilized in the case of beads, complex determined either directly or indirectly, for example, as described above. Alternatively, the complexes can be dissociated from the matrix, and the level of CEA binding or activity determined using standard techniques. Other techniques for immobilizing proteins on matrices (and well-known in the art) can also be used in the screening assays of the invention. Conjugation of biotin and streptavidin, is but one example of an immobization technique. Alternatively, antibodies reactive with CEA but which do not interfere with the CEA-CEA binding regions of the present invention can be derivatized to the wells of the plate, and unbound CEA protein trapped in the wells by antibody conjugation. Methods for detecting such complexes, in addition to those described above for the GST-immobilized complexes, include immunodetection of complexes using antibodies reactive with the CEA.

As used herein, the term “physical association” refers to an interaction between two moieties involving contact between the two moieties.

As used herein, the term “fusion protein(s)” refers to a protein encoded by a gene comprising amino acid coding sequences from two or more separate proteins fused in frame such that the protein comprises fused amino acid sequences from the separate proteins.

As used herein, the term “host cell(s)” is a cell which has been transformed or transfected, or is capable of transformation or transfection by an exogenous polynucleotide sequence.

As used herein, the term “immunospecific” means that characteristic of an antibody whereby it possesses substantially greater affinity for the polypeptides of the invention or the polynucleotides of the invention than its affinity for other related polypeptides or polynucleotides respectively, particularly those polypeptides and polynucleotides in the prior art.

As used herein, the term “recombinant expression system(s)” refers to a system in which vectors comprising sequences encoding polypeptides of the invention or portions thereof, or polynucleotides of the invention are introduced or transformed into a host cell or host cell lysate for the production of the polynucleotides and polypeptides of the invention.

As used herein, the term “artificially synthesized” when used in reference to a peptide, polypeptide or polynucleotide means that the amino acid or nucleotide subunits were chemically joined in vitro without the use of cells or polymerizing enzymes. The chemistry of polynucleotide and peptide synthesis is well known in the art.

In addition to the standard single and triple letter representations for amino acids, the term “X” or “Xaa” may also be used in describing certain polypeptides of the invention. “X” and “Xaa” mean that any of the twenty naturally occurring amino acids may appear at such a designated position in the polypeptide sequence.

As used herein, the term “specifically binding” in the context of the interaction of two polypeptides means that the two polypeptides physically interact via discrete regions or domains on the polypeptides, wherein the interaction is dependent upon the amino acid sequences of the interacting domains. Generally, the equilibrium binding concentration of a polypeptide that specifically binds another is in the range of about 1 mM or lower, more preferably 1 uM or lower, preferably 100 nM or lower, 10 nM or lower, 1 nM or lower, 100 pM or lower, and even 10 pM or lower.

As used herein, the term “decrease in the binding” refers to a drop in the signal that is generated by the physical association between two polypeptides under one set of conditions relative to the signal under another set of reference conditions. The signal is decreased if it is at least 10% lower than the level under reference conditions, and preferably 20%, 40%, 50%, 75%, 90%, 95% or even as much as 100% lower (i.e., no detectable interaction).

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand).

As used herein, the term “purified” refers to a molecule having been separated from other cellular components. Thus, for example, a “purified protein” has been purified to a level not found in nature. A “substantially pure” molecule is a molecule that is not contaminated with most other cellular components.

As used herein, the terms “molecule”, “compound”, “agent” or “ligand” are used interchangeably and broadly to refer to natural, synthetic or semi-synthetic monovalent molecules or compounds. The term “molecule” therefore denotes for example chemicals, macromolecules, cell or tissue extracts (from plants, microorganisms, or animals) and the like. Non-limiting examples of molecules include nucleic acid molecules, peptides, antibodies, carbohydrates and pharmaceutical agents. The agents can be selected and screened by a variety of means including random screening, rational selection and by rational design using for example protein or ligand modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the terms “molecule” or “agent”. For example, peptidomimetics, well known in the pharmaceutical industry and generally referred to as peptide analogs, can be generated by modeling as mentioned above. Similarly, in a preferred embodiment, the polypeptides of the present invention are modified to enhance their stability. It should be understood that in most cases this modification should not alter the biological activity in inhibiting the CEA-mediated tumorigenic effects. The molecules identified in accordance with the teachings of the present invention have a therapeutic value in diseases or conditions in which the physiology or homeostasis of the cell and/or tissue is compromised by CEA-mediated clustering (i.e., tumorigenic effects). Alternatively, the molecules identified in accordance with the teachings of the present invention find utility in the development of more effective releasers of CEA-mediated tumorigenic effects or more effective declustering agents. Such agents can be further used or identified using assays of the present invention. In addition, the targeted region of CEA can be used to design further ligands which span CEA domains involved in CEA-CEA interactions and in particular CEA regions involved in clustering. Non-limiting examples of such CEA region include the N-terminal region of CEA and more particularly the three subdomains thereof (GYSWYK, NRQII, and QND, as well as regions comprising same or flanking same or linking same which are involved in CEA-CEA interactions and especially in CEA clustering). Methods to identify epitopes, to design monovalent ligands such as peptides and their derivatives, or monovalent antibodies and their derivatives are well-known in the art.

Composition within the scope of the present invention should contain the monovalent binding agent in an amount effective to achieve the desired therapeutic effect (e.g. declustering effect) while avoiding adverse side effects. Typically, agents in accordance with the present invention can be administered to mammals (e.g. humans) in doses ranging from 0.001 to 50 mg per kg of body weight per day of the mammal which is treated. Pharmaceutically acceptable preparations and when applicable salts of the active agent are within the scope of the present invention and are well known in the art (Remington's Pharmaceutical Science, 16th Ed., Mack Ed.). The dosage will be adapted by the clinician in accordance with conventional factors such as the extent of the disease and different parameters from the patient. It will be understood that more than one agent of the present invention can be combined. In addition, other anti-cancer agents could be combined with the monovalent binding agent of the present invention.

Abbreviations used herein are: CAM, cell adhesion molecule; IgSF, immunoglobulin superfamily; CEA, carcinoembryonic antigen; NCAM, neural cell adhesion molecule; GM, growth medium; DM, differentiation medium; FITC, fluorescein isothiocyanate.

The external domains of Immunoglobulin Superfamily (IgSF) members are involved in multiple binding interactions, both homophilic and heterophilic, that initiate molecular events leading to the execution of diverse cell functions. Human carcinoembryonic antigen (CEA), an IgSF cell surface glycoprotein used widely as a clinical tumor marker, undergoes homophilic interactions which mediate intercellular adhesion. Recent evidence supports the view that deregulated over-expression of CEA has an instrumental role in tumorigenesis by the inhibition of cell differentiation and the disruption of tissue architecture. The CEA-mediated block of the myogenic differentiation of rat L6 myoblasts depends on homophilic binding of its external domains. In addition, the role of CEA interactions in tumorigenesis has been well described.

The structural requirements for the differentiation-blocking activity of CEA, using the myogenic differentiation of rat L6 myoblasts as a model system, were investigated with the goal of designing agents that were capable of releasing this activity. The basic approach was to identify small domains whose integrity was essential for the function, then to test the effect of agents which could interfere with their interaction. mAb monovalent Fab fragments that bind to them were used as non-limiting examples of such agents. Three such subdomains were identified as validated target sites and monovalent binding agents such as cyclized peptides and mAb Fab fragments were found to be effective in releasing the CEA differentiation block in L6 myoblasts, and in therefore reducing tumorigenicity.

The rationale used herein for the structure-function aspect of the present invention was based on previous results showing that the N and A3B3 domains of CEA were required for the differentiation blocking effect (20), thus implicating the involvement of double reciprocal bonds between anti-parallel molecules on apposite cell surfaces, as observed for the intercellular adhesion function of CEA (9). A direct experiment showing that L6 (CEA) transfectants could “trans-block” the differentiation of differentiation-competent L6 (ΔNCEA) transfectants supports the notion that anti-parallel CEA-CEA interactions between cells can contribute to the myogenic differentiation block. Parallel CEA-CEA interactions, probably on the same cell surface, were also implicated by the demonstration that the deletion mutant ΔNCEA, normally incapable of mediating the differentiation block, could be rendered capable of blocking L6 differentiation by cross-linking with specific antibodies. Although parallel interactions can be envisioned to improve intercellular adhesive forces by clustering anti-parallel-interacting inter-cellular molecular pairs (the velcro effect) (13), their relative contribution could be greater for the differentiation blocking function (32). These considerations may underlie the observation that the intercellular adhesion and differentiation-blocking functions of CEA could be separated by certain amino acid substitutions in subdomain 3 that removed the differentiation-blocking activity while leaving the intercellular adhesive activity intact. This result could therefore be interpreted by the suggestion that subdomain 3 is primarily necessary for parallel binding between CEA molecules. Consistent with this, peptide QNDTG, representing the 3^{rd} subdomain, was the most effective in releasing the CEA-imposed differentiation block.

CEA expression inhibits molecular events occurring very early in the myogenic differentiation process, notably the upregulation of the myogenic transcriptional regulator, myogenin (20). Recent evidence indicates that the molecular basis for the pan-inhibition of cellular differentiation mediated by CEA involves perturbation of the function of certain integrins (α₅β₁ in L6 myoblasts and human colonocytes) (Ordoñez et al., submitted), which are known to affect the earliest steps in differentiation (33-35).

The results reported here, showing the involvement of subdomains of the CEA molecule required for CEA-CEA binding, favor a clustering model in which CEA and specific integrins inhabit the same membrane rafts defined by the GPI anchor of CEA. In such a model, clustering of CEA would thereby cause clustering of integrin molecules, which in turn would result in a change in integrin function (36). Clustering of CEA would be expected to be effected mainly by parallel interactions on the same cell surface but antiparallel interaction may be required to initiate the process of clustering. This model explains why monovalent Fab fragments of specific mAbs were found to be required to release the CEA-mediated differentiation block. Whole divalent mAbs have the effect of enhancing clustering and would thus be expected to increase the tumorigenic effects of CEA.

With the knowledge that there are subdomains in the CEA molecule that can be differentially antagonized to affect the differentiation-blocking function and decrease the CEA-mediated tumorigenic effects, while leaving the intercellular adhesive function intact, the possibility of designing agents with functionally selective blocking activity can be entertained. Subdomain 3 was identified as such a region but, while its corresponding peptide was effective in releasing the CEA-imposed differentiation block, it was also effective in inhibiting CEA-mediated intercellular adhesion (16). Thus, although QNDTG appears to represent an example of an experimentally useful agent with potential for medical application, further application of the methods outlined herein is expected to yield even more potent and selective agents. Indeed, the agents of the present invention as well as others can be modified and tested utilizing the methods of the present invention to assess their potency and selectivity. In any event, the present invention already provides effective and selective monovalent binding agents which significantly release the CEA-mediated differentiation block and importantly significantly decrease the tumorigenicity of CEA-mediated interactions in vitro and in vivo.

Finally, even though the results to date have shown that the L6 myogenic differentiation model system used here is accurately predictive of results obtained with more medically relevant systems, the findings bear repeating in other less experimentally convenient systems such as human Caco-2, LS-180 and SW-1222 colonocytes, in which several CEA family members are normally expressed in a regulated fashion (37). In the latter cells, deregulated over-expression of both CEA and CEACAM6 at levels observed in freshly excised human tumor colonocytes, has been shown to block cell polarization and disrupt tissue architecture (16). Present findings indicate that Fab fragments of mAbs A20 and B18 are capable of reversing these tumorigenic effects (42), thus providing an appealing strategy for the reversal of the malignant phenotype in medically relevant situations. The validation of the agents identified herein and of their relevance to differentiation, proliferation and cancer is shown herein below (FIGS. 10-12).

The myogenic differentiation block requires the CEA-specific glycophospholipid inositol (GPI) anchor determined by the processed carboxy-terminal sequence of CEA, together with extracellular domains that can self-bind (such domains from an irrelevant molecule will suffice (40)). It is suggested that the CEA GPI anchor determines a membrane raft that also contains the integrin α5β1. Because of the self binding of the external domains of CEA, the CEA-containing rafts cluster on the cell surface, thus clustering the integrin molecules as well (FIGS. 13 and 14). Such clustering activates the integrin molecules, setting off a chain of signaling events that eventually lead to inhibition of differentiation and anoikis [Ordonez, et al., 2000, Cancer Research 60, 3419], a blocking of cell polarization and a disruption of tissue architecture.

Both intercellular adhesion [Taheri et al., 2000, J. Biol. Chem. 275, 26935] and the myogenic differentiation block of rat L6 myoblasts are shown herein by site-directed mutation to require 3 specific subdomains in the N domain of CEA: G₃₀YSWYK, N₄₂RQII and Q₈₀NDTG.

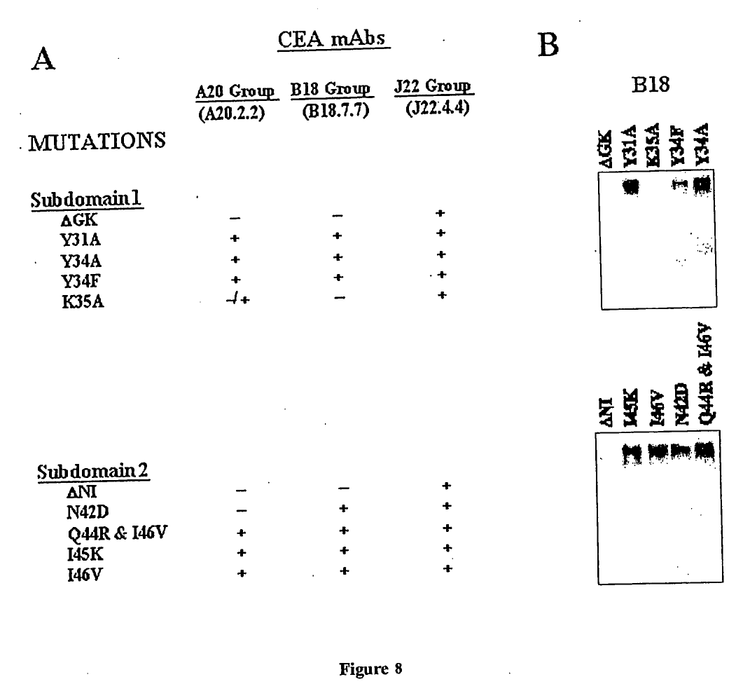

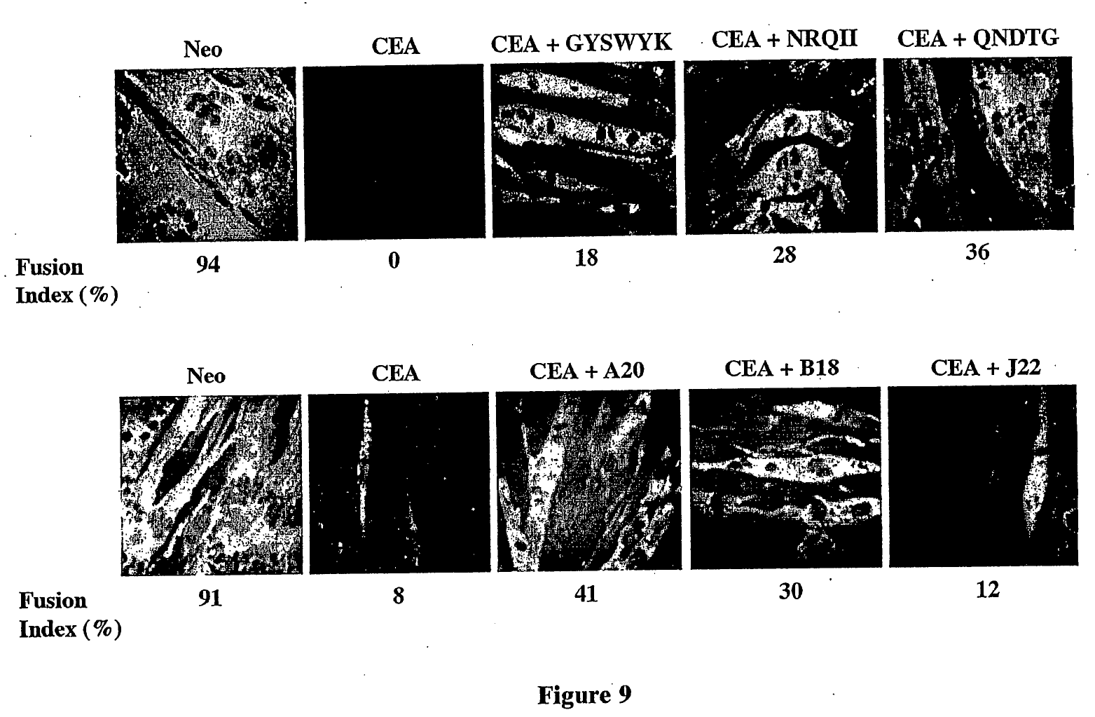

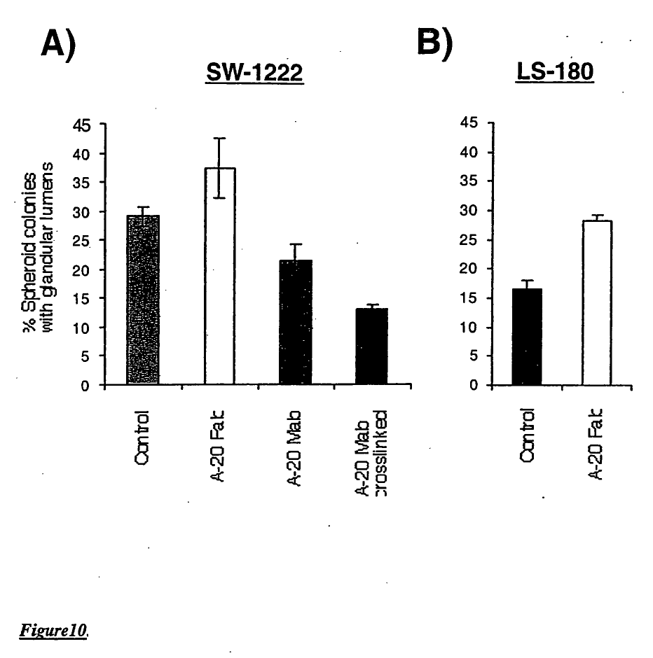

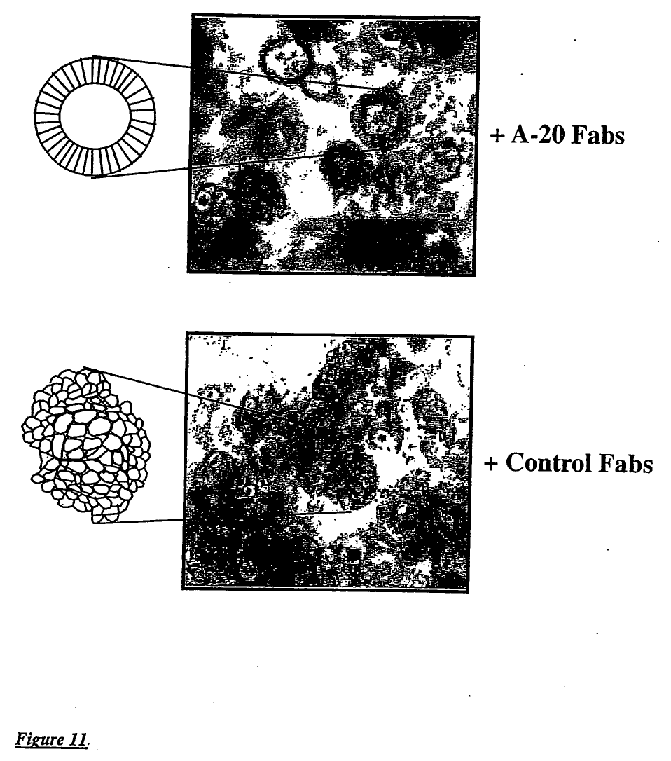

Monoclonal antibodies A20 (16) and B18 (FIG. 8) are shown to bind to epitopes shifted about one amino acid residue relative to each other, both bridging GYSWYK and NRQII. Monovalent Fab fragments of both mAbs were demonstrated to partially release the CEA-imposed myogenic differentiation block giving 30-40% myogenic differentiation (FIG. 9). Of note, whole divalent A20 and B18 mAbs had no effect on the differentiation block (data not shown). These results support the contention that CEA molecules must be clustered on the cell surface in order to exert their tumorigenic effects, thereby validating the clustering model given above. Monovalent Fab fragments of mAbs that bind to the key subdomains (or bridging regions there between) required for these affects have the effect of breaking up CEA clusters, while divalent whole mAbs, tend to keep them together and even to cluster any free CEA molecules that might exist on the cell surface. The present invention is not limited to the exact epitopes or peptides exemplified in the present invention, since these epitopes or peptides can be shifted by a few amino acids provided that the CEA-CEA interaction domains responsible for CEA-mediated differentiation block and/or CEA-mediated tumorigenic effects is targeted. It should be clear that shifting the targeted epitopes by one or more amino acid can be carried out in accordance with the present invention. It should also be understood that it can be predicted that a shifting by more amino acids will show tumorigenic reversing effects and/or differentiation block reversing effects, provided that the shifted targeted region directly or indirectly influences the CEA-interactions responsible for the block or effects described herein. Means of designing and testing further monovalent ligands are amenable to the person of ordinary skill. It will be understood that a direct effect means an effect due to a targeting of a region of CEA directly implicated in CEA interaction. An indirect effect is meant to cover a targeting of a region which is not necessarily involved in the CEA interaction, but which nevertheless influences this interaction (by affecting folding or by steric hindrance of self binding for example).

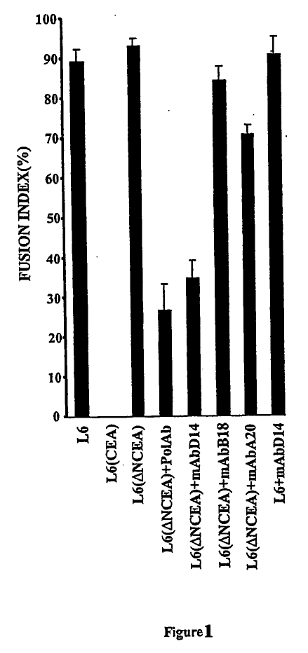

Studies utilizing a deletion mutant of CEA, deltaNCEA, that lacks all three of the above subdomains, further substantiate these claims. This mutant is naturally completely defective in mediating either intercellular adhesion or the myogenic differentiation block. Because of a lack of self-binding, these molecules cannot cluster on the cell surface, despite being present at relatively high concentrations. When induced to cluster artificially, however, by cross-linking with whole divalent mAb D14 (that binds to an epitope in the A3 domain of CEA still present in deltaCEA) or with whole polyclonal anti-CEA Abs, the deltaNCEA molecules now become capable of blocking myogenic differentiation (FIG. 1).

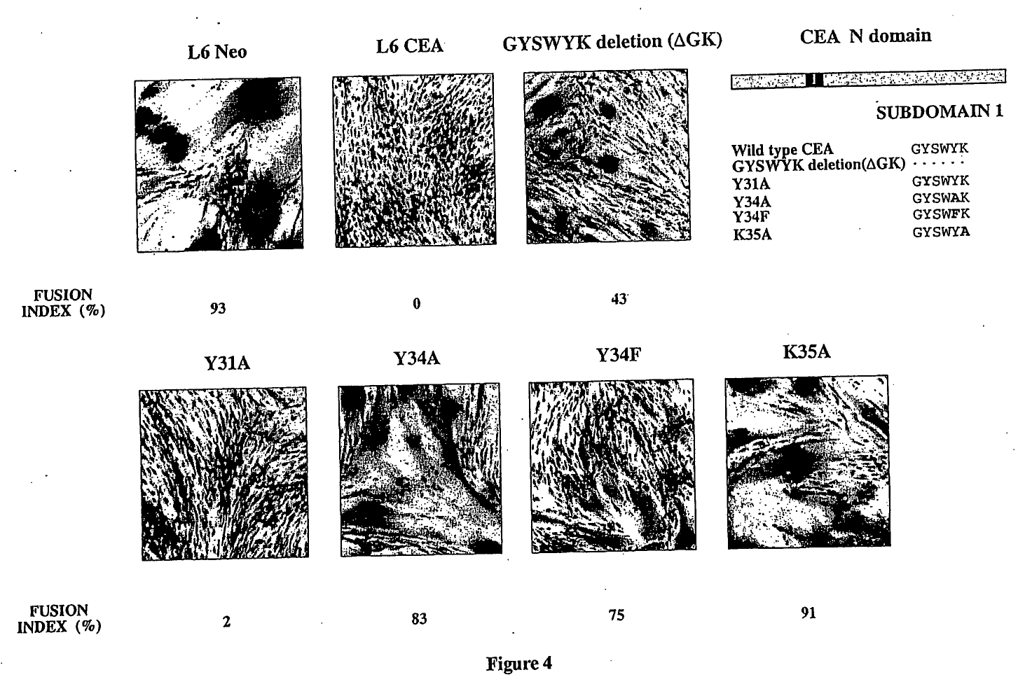

Furthermore, one of the very early events, the activation of Integrin-Linked Kinase (ILK), is apparent within 5 minutes of cross-linking the deltaNCEA molecules on the surface of L6 cells or on human colonocyte Caco2 cells (FIG. 4). The activation of this kinase is believed to result from the clustering of the α5β1 integrin and is an event required for the execution of signaling pathways leading to the tumorigenic effects of CEA on the cellular phenotype (and leading to integrin activation). In fact, within 2-5 minutes of cross-linking the deltaNCEA molecules on the surface of L6 cells integrin α5β1 is activated, as evidenced by the increased binding of its major ligand, fibronectin (—FIG. 17).

The present invention is illustrated in further detail by the following non-limiting examples.

### EXAMPLE 1

### Experimental Procedures

Materials—Cyclized and linear-blocked oligopeptides were obtained (>95% purity) from Multiple Peptide Systems (San Diego, Calif.). Linear-blocked peptides were rendered more stable by acetylating the amino terminus and aminating the carboxy terminus. Cyclic peptides contained two cysteine residues joined by sulfide bonds at their termini. The peptides used were blocked linear NAc-LFGYSWYKGE-NH2, NAc-VDGNRQIIGY-NH2, NAc-RIIQNDTGFY-NH2 and NAc-FNVAEGKEV-NH2; and cyclized H-CGYSWYKC-OH, H-CGNRQIIC-OH, H-CQNDTGC-OH and H-YCTDEKQCY-OH, representing subdomains 1, 2 and 3, and control peptides, respectively. Sequences actually present in the N domain of CEA for the cyclized peptides are underlined.

Construction of CEA cDNA Mutants—Wild type cDNA coding for CEA (17) was used as a template for all polymerase chain reaction-generated (PCR) constructs. The recombinant PCR technique (25) was used to generate site-directed mutants as described previously (16).

Cell Culture—Rat L6 myoblasts were grown as monolayer cultures at 37° C. in a humidified atmosphere with 5% CO₂ in Dulbecco's modified Eagle's medium (DMEM) containing 10% fetal bovine serum (FBS, Invitrogen), 100 U/ml penicillin and 100 μg/ml streptomycin (Invitrogen) (growth medium; GM). Cell cultures were subcultured while subconfluent to avoid selection of non-fusing variants. LR-73 cells (26), derived from the CHO line, were grown in monolayer culture in αMEM (27) containing 10% FBS, 100 U/ml penicillin and 100 μg/ml streptomycin, at 37° C. in a humidified atmosphere with 5% CO₂.

Transfection—L6 myoblast cells were seeded at 2×10⁵ cells/100 mm plastic tissue culture Petri dish and cotransfected 24 hr later by the calcium phosphate-mediated co-precipitation method, as described (21), with 5 μg of p91023B expression vector containing CEA (WT or mutant) cDNA, 10 μg of LR-73 carrier genomic DNA and 0.5 μpg pSV2neo plasmid per dish. Stable pooled transfectant colonies were isolated by selection with 400 μg/ml Geneticin (G418, Gibco-BRL, Grand Island, N.Y.). Immunofluorescent labeling with anti-CEA monoclonal antibody J22 (29) and FACS sorting was carried out to select for populations of transfectants stably expressing desired levels of mutant or wild type CEA on the cell surface. At least 2 independent pooled populations of transfectant clones were isolated for each transfected cDNA. All transfectant populations were maintained in growth medium containing 400 μg/ml of G418. G418 was removed from the medium 24 hours before each functional assay was performed.

FACS Analysis—Cells were removed from culture vessels by light trypsinization (a treatment that does not affect cell surface levels of CEA) and resuspended in ice cold PBS+2% FBS (PBSF). 2.5×10⁵ cells were incubated with polyclonal rabbit or monoclonal anti-CEA antibodies (J22) at a dilution of 1:100 in PBSF for 35 min on ice. Cells were washed with 2.5 ml PBSF, centrifuged, and resuspended in 0.5 ml PBSF containing FITC-conjugated goat anti-rabbit or anti-mouse antibody at a dilution of 1:100. After 30 min incubation on ice, cells were washed, centrifuged, and resuspended in 0.75 ml PBSF and analyzed using a Becton Dickinson FACScan® instrument (Bedford, Mass.).

Adhesion Assays—LR-73 cells were seeded at 1×10⁶ per 80 cm² culture flask (Nalge Nunc Inc., Naperville, Ill.) in LR-73 growth medium. After two days in monolayer culture, the cultures were rendered single cell suspensions by 3 min incubation at 37° C. with 0.12% Bacto trypsin in PBS lacking Mg²⁺ and Ca²⁺ and containing 15 mM sodium citrate. The cells were incubated at 10⁶ cells/ml in α-MEM containing 0.8% FBS and 10 μg/ml of DNase I at 37° C. with stirring at 100 rpm (28). The percentage of cells remaining as single cells, which declines over time due to formation of aggregates, was determined as a function of time by visual counting using a hemocytometer.

Differentiation Assays—To initiate fusion and differentiation, L6 cultures were seeded at 10⁴ cells/cm² at day 0 in 60 mm or 35 mm tissue culture Petri dishes or 7×10³ cells/cm² in multiwell plastic chamber slides (Nalge Nunc Inc., Naperville, Ill.) and grown in GM. The medium was replaced after 3 days with D-MEM+2% horse serum (differentiation medium; DM) and the cells cultured for an additional 5-7 days. To co-culture L6 (CEA) and L6 (ΔNCEA) cell transfectants, cells were seeded at 3×10⁵ cells of each type per 35 mm plastic tissue culture Petri dish in GM; 24 hr later the medium was replaced with DM. For fusion index determinations, cells were fixed with 2.5% glutaraldehyde and stained with hematoxylin. The fusion index was calculated as the percentage of total nuclei contained in fused myotubes having more than 3 nuclei per myotube, as described previously (20). Fusion determinations were repeated 3 times (independent experiments) for each of 2 independently obtained transfectant populations for each mutant. The values reported in FIGS. 4, 5, and 6 represent the averages of these determinations.

As a biochemical measure of myogenic differentiation, cells were fixed in methanol: acetone (3:7) at −20° C., and processed for immunofluorescent staining with anti-myosin mAb (30).

To study the effect of cyclized or linear-blocked peptides on the CEA-mediated L6 differentiation block, cells were seeded at 7×10³ cells/cm² in 8 well chamber slides (Nalge Nunc™ Inc., Naperville, Ill.) in GM on day zero. After 3 days' incubation, the medium was replaced with DM containing peptide at the indicated concentrations.

Purification of Antibodies—Rabbit polyclonal anti-CEA antibody and mouse monoclonal anti-CEA antibodies (A20, B18, and D14) were purified with Bio-rad Affi-Gel™ protein A MAPS™ II kit (Bio-Rad Laboratories, Hercules, Calif.). Antibodies were added to differentiation medium to a final concentration of 1 mg/ml. Fab fragments of monoclonal anti-CEA antibodies were prepared as described previously (29). Fab fragments were added to differentiation medium to a final concentration of 100 μg/ml. The values for fusion indices shown in FIG. 9 represent the averages of 3 independent determinations.

### EXAMPLE 2

### Nature of CEA Homophilic Intermolecular Interactions Required for Differentiation Block

To test for a role of parallel CEA-CEA interactions on the same cell surface, differentiation-competent L6 (ΔNCEA) transfectants were treated with cross-linking polyclonal and monoclonal anti-CEA antibodies. Antibodies for which the binding epitopes are still intact in the ΔNCEA molecule, rabbit polyclonal and D14 [binding epitope at the B2-A3 junction (29)], converted ΔNCEA to a differentiation-blocking molecule, whereas control antibodies directed to binding epitopes that are missing in ΔNCEA, A20 and B18, two N-domain specific mAbs, [binding epitopes at residues 35 to 42, in the N domain (16)] were without effect (FIG. 1). To control for non-specific effects, one of the effective antibodies, D14, was shown to have no effect on the differentiation of the parental L6 cells (FIG. 1).

These experiments and experiments showing the efficacy of trans-blocking support the hypothesis that both anti-parallel and parallel binding between CEA molecules are involved in the CEA-mediated myogenic differentiation block. Furthermore and of relevance, these results further support the clustering model.

### EXAMPLE 3

### Structural Requirements for Myogenic Differentiation Blocking Function of CEA

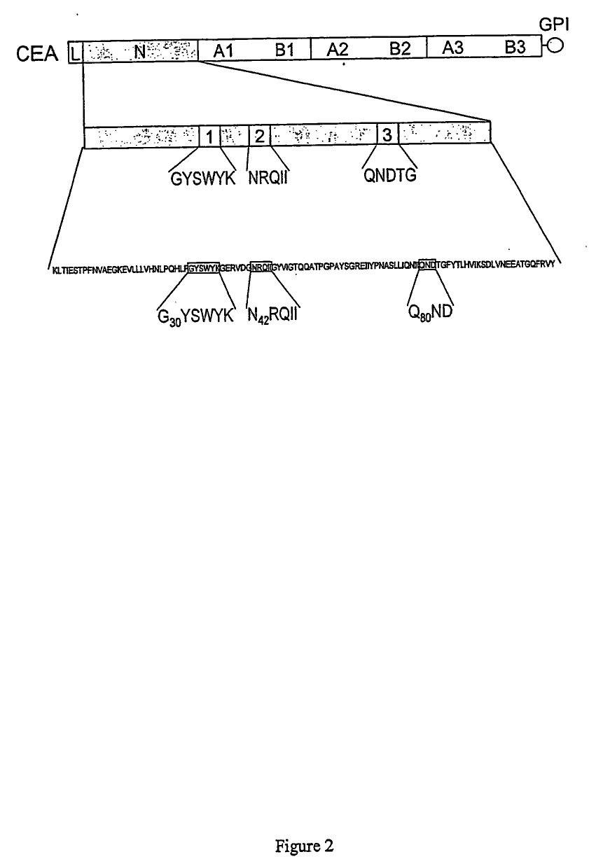

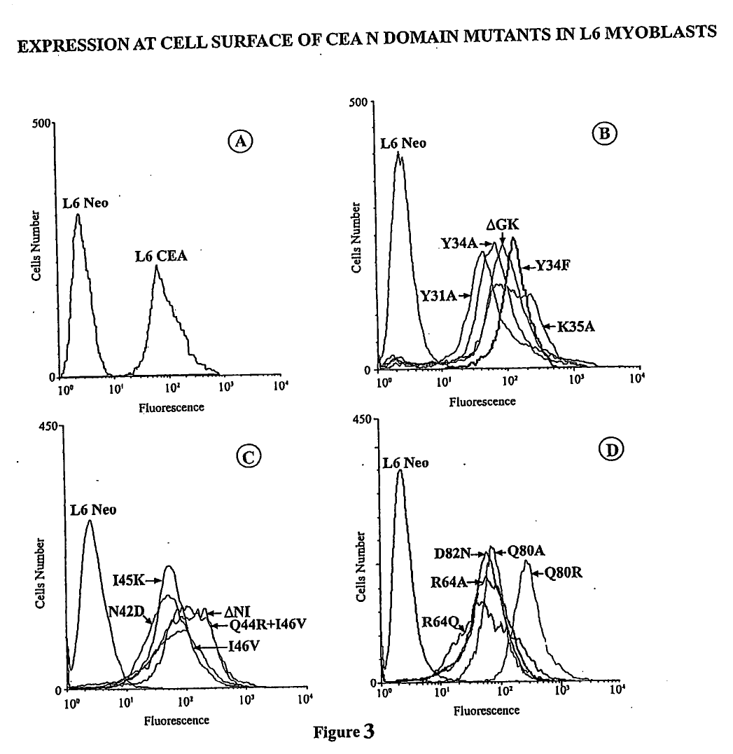

Deletions and substitutions in three subdomains of the N domain of CEA (FIG. 2) were produced by site-directed mutagenesis, as described previously (16). The rationale for choosing these particular subdomains can be summarized as follows: the requirement for N domain amino acids 32 to 106, deleted in mutant ΔNCEA, for the myogenic differentiation block was demonstrated previously (20). Within this deletion, subdomains 1 and 2 were implicated by the fact that (1) monovalent Fab fragments of mAb A20 can release the CEA-imposed myogenic differentiation block and reverse the CEA-mediated tumorigenic effect (see below) and has a binding epitope that bridges them; (2) this epitope includes the carboxy-terminal amino acid of subdomain 1 and the amino-terminal amino acid of subdomain 2 (16) and (3), these subdomains (1 and 2) and subdomain 3 were all shown to be important in CEA-mediated intercellular adhesion in LR-73 cells. Finally, and indicative of their involvement in CEA-CEA interactions, all three subdomains were demonstrated to be adjacent and exposed in a 3-dimensional structural model based on the known structures of CD2 (16) and of CD4 (Saragovi, unpublished). Pooled stable transfectant clones of L6 cells expressing comparable cell surface levels, as assessed by FACS analysis (FIG. 3), were isolated for each of the deletion and substitution mutants.

Concerning subdomain 1, G₃₀YSWYK, substitutions at the carboxy terminus, Y34A, the more conservative Y34F, and K35A, had a profound effect on the myogenic differentiation-blocking activity of CEA, while mutation Y31A at the amino terminus had no effect (FIG. 4). As expected, deletion of the entire subdomain 1 (ΔGK) also affected this function of CEA but, curiously, was not as effective as substitutions Y34A and K35A.

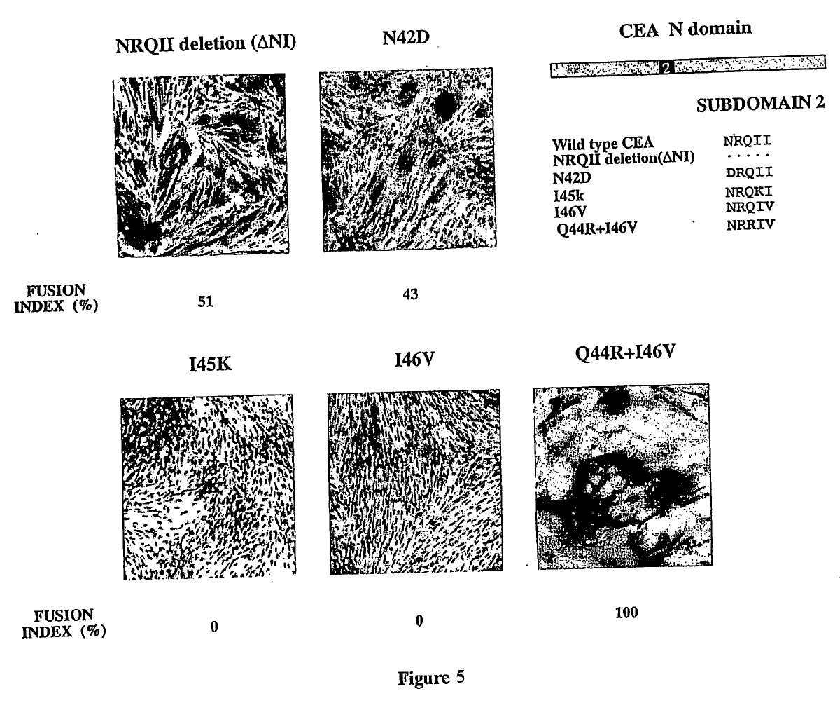

Similarly, the deletion of subdomain 2, N₄₂RQII, had less effect on CEA function than some of the substitutions within this domain, notably the double mutation Q44R+I46V in subdomain 2, for which the degree of differentiation of 100% actually exceeded that of parental L6 cells, with a reproducible effect (FIG. 5). A single mutation at the amino terminus of the subdomain, N42D, partially removed the differentiation-blocking activity of CEA.

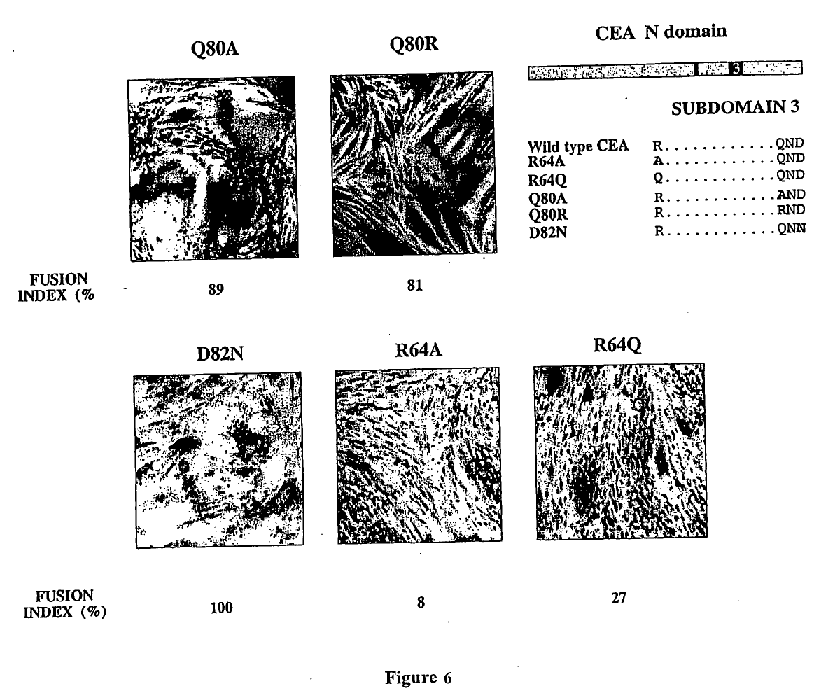

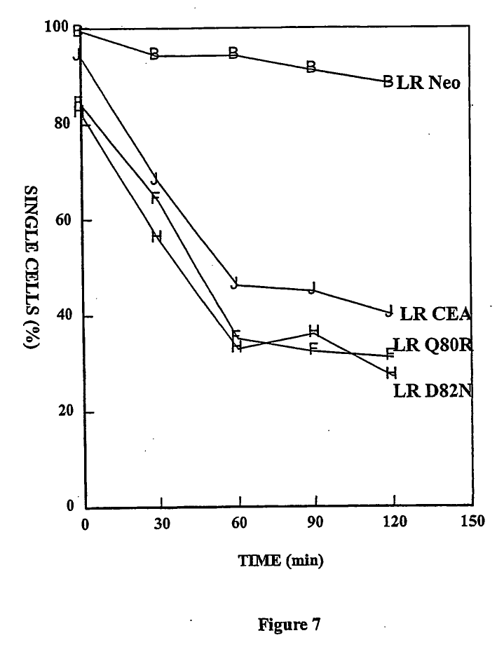

The third subdomain, Q₈₀NDTG, was found to play a critical role in the CEA-mediated differentiation block, since mutation Q80A resulted in a complete loss of this function and mutation D82N, like Q44R+I46V in subdomain 2, gave 100% differentiation, thus exceeding that of the parental cells (FIG. 6). Mutations Q80R, giving 81% differentiation and D82N, giving 100% differentiation are of particular interest since Q80R and D82N, if anything, actually enhanced the intercellular adhesion function of CEA expressed in LR-73 cells (FIG. 7). These mutations therefore separate the intercellular adhesion and differentiation blocking functions of CEA.

Two other mutations at residue R64, a site shown by Sippel et al. (31) to have a marked effect on the intercellular adhesion function of CEA expressed in insect cells, had relatively small effects on the differentiation blocking function of CEA in rat L6 myoblasts (FIG. 6).

### EXAMPLE 4

### Effects of Monovalent Monoclonal Anti-CEA Antibodies on CEA-mediated Differentiation Block

The epitope of the adhesion-inhibitory anti-CEA mAb A20 (29) was shown previously to bridge the carboxy-terminal K₃₅ residue of the first subdomain and the amino-terminal N₄₂ residue of the second subdomain (16). The binding epitope of anti-CEA mAb B18, which also inhibits intercellular adhesion (although less effectively than A20—data not shown), was found to be shifted slightly upstream from that of A20, in that the K35A mutation completely abrogated B18 binding but, unlike A20 (16), the N42D mutation was without effect (FIG. 8). Binding to CEACAM8, which differs from all other CEA family members by the presence of an Ala versus a Gly residue at position 41, was completely absent in both B18 and A20 (data not shown) and binding to Y34F was reduced somewhat for both (FIG. 8 and (16)). All other point mutations in the first two subdomains [FIG. 8 and (16)] and the third subdomain (data not shown) were without effect.

The effects of monovalent Fab fragments of A20 and B18 on the CEA-mediated myogenic differentiation block are shown in FIG. 9. Both partially released the block and, as in their effects on intercellular adhesion, A20 was more effective than B18, giving an average value of 41% fusion at a concentration of 100 μg/ml in 3 independent experiments. Once again, whole divalent mAbs were without effect on the differentiation block (data not shown), thereby reinforcing the validation of the clustering model and the importance of inhibiting, abrogating or reversing CEA-dependent clustering and its associated differentiation block and tumorigenic effects.

### EXAMPLE 5

### Effects of Peptides on CEA-Mediated Differentiation Block

In order to test other ligands which could interfere with the CEA sub-domains interactions responsible for the myogenic block, peptides representing the subdomains 1 to 3 of CEA were tested to asses whether they could release the CEA-imposed myogenic differentiation block. Peptides both terminally blocked (for improved stability) and cyclized (for both improved stability and conformation) were tested by addition to L6 (CEA) cells cultured in the presence of DM. All linear peptides were virtually ineffective (data not shown) but were effective when cyclized (FIG. 10), a conformation which is expected to mimic a β-turn configuration in the native molecule, the configuration predicted for the 3 subdomains. Maximum activity in releasing the differentiation block was found at a concentration of about 100 μM; higher concentrations of the peptides were less effective due to non-specific toxicity. At 100 μM, cyclized peptide QNDTG released the myogenic differentiation block to the greatest extent, with an average of 36% fusion in 3 experiments versus 28% and 18% for cyclized peptides NRQII and GYSWYK, respectively, relative to 94% for control L6 (Neo) myoblasts. Experiments done with combinations of the three different peptides showed that there was synergy between NRQII and QNDTG; lower concentrations of these peptides (40 μM of each) had the same effect as 100 μM of QNDTG (data not shown).

### EXAMPLE 6

### Release of the CEA-Mediated Differentiation Block in Human Colorectal Carcinoma Cells Using Monovalent Antibodies In Vitro

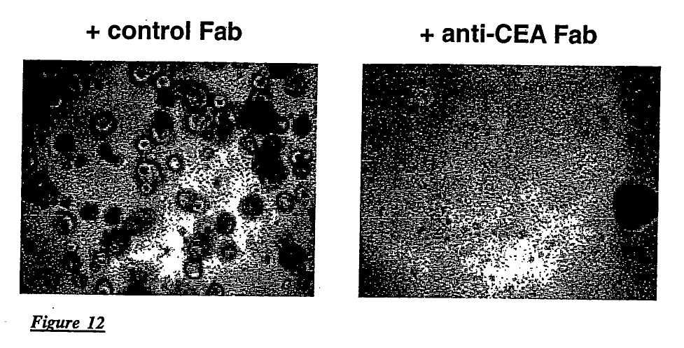

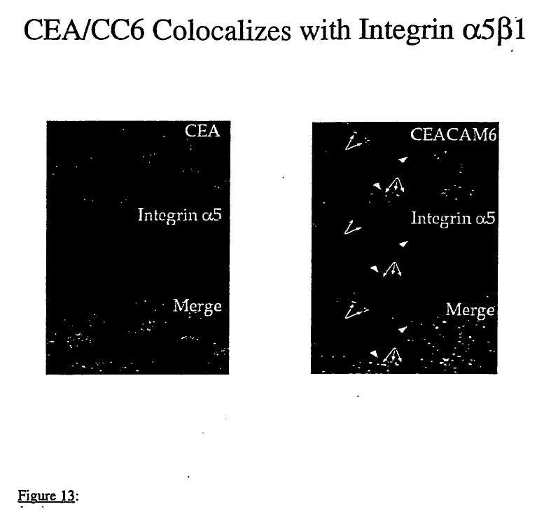

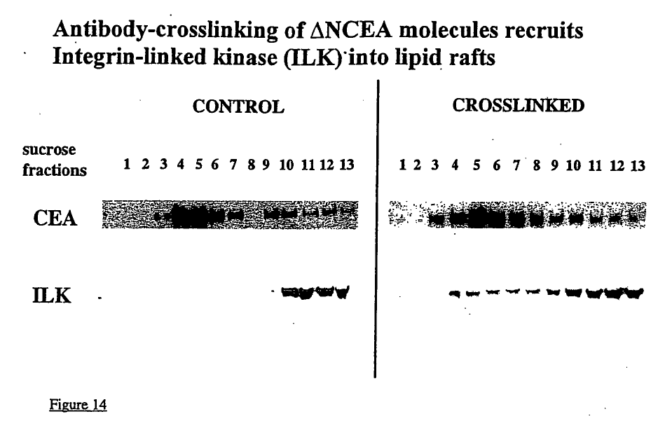

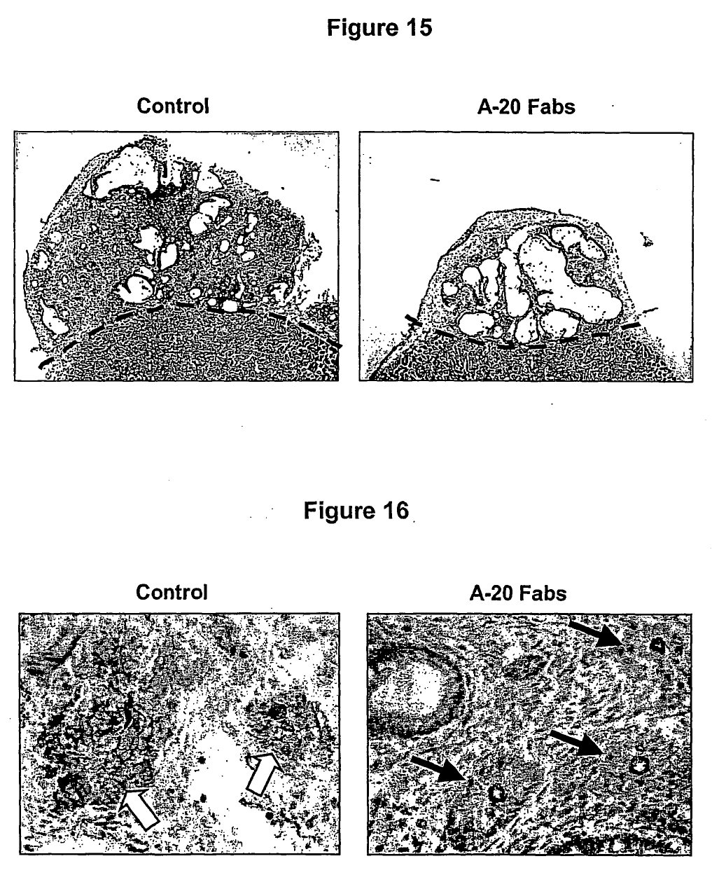

It has been shown previously that deregulated over-expression of CEA and CEACAM6 in human colonocyte cell lines can block cell polarization and inhibit anoikis, thus disrupting tissue architecture (41; Stanners et al, WO 99/41370). One of the assays employed was measurement of the ability of human colorectal carcinoma cell line, SW-1222, to form gland-like spheroids resembling closed colonic crypts in collagen gels. FIG. 10 shows that the application of whole mAb A20 to SW-1222 cells actually leads to fewer spheroids with central lumens and gland-like architecture than untreated SW-1222 cells, and cross-linking of the bound A20 with a secondary anti-mouse IgG antibody leads to even fewer well differentiated spheroids. In contrast, treatment of SW-1222 cells with Fab fragments of A20 gives a higher frequency of well differentiated spheroids (FIG. 10). The latter normalizing effect of Fab fragments was better shown with LS-180, another human colorectal carcinoma cell line. This line has very low ability to form gland-like spheroids, forming mainly featureless tumor-like cell collectives in collagen gels. Treatment of LS-180 cells with A20 Fab fragments resulted in a significant proportion of well-differentiated spheroids (FIG. 11). In some experiments, treatment with Fab fragments actually resulted in complete loss of the ability of LS-174T/SW-1222 cells to form any spheroids, indicating reversal to a state of normalcy that is completely incapable of supporting anchorless growth in suspension in collagen gels (FIG. 12 and Table 1). This treatment resulted in a frequency of spheroids that was less than about 1% of that obtained with Fab fragments of control antibody (see Table 1, for quantitation).

TABLE 1
number of tumor spheroids in anti-CEA treated wells
versus control Fab treatment

| | +anti-CEA Fab | +control Fab |
|---|---|---|
| Cell line | # of spheroids† | # of spheroids |
| LS-174T | 13 | 1661 |
| SW-1222 | 11 | 2000 |

†average of two wells

These results are consistent with the proposed model for the tumorigenic effects of CEA and CEACAM6 [Screaton et al., 2000, J. Cell Biol. 150: 613-625]. Without being limited to a particular theory, in this model, the effects of CEA are proposed to be due to clustering of CEA molecules on the cell surface due to self-binding of their external domains. Divalent whole mAbs would be expected to link CEA molecules together, thus increasing clustering and increasing tumorigenic effects, and secondary cross-linking antibodies would be expected to augment the effect, as observed. Monovalent ligands as exemplified with the non-limiting Fab fragments of mAbs that bind to epitopes involved in CEA interaction (e.g. self-binding), on the other hand, have only one binding site and so should actually block self binding and should thus have the desired effect of preventing clustering, thus reducing CEA and CEACAM6's tumorigenic effects. This is what is presented herein, thereby fully supporting this clustering model, not only in vitro, but also in vivo (see below).

### EXAMPLE 7

### Efficacy of Fabs Applied to Human Colorectal Carcinoma Cells In Vivo

In order to assess whether CEA-monovalent agents can reverse the CEA-mediated tumorigenic effects in vivo (and whether the CEA-mediated differentiation block can also be released in vivo), in vivo experiments were carried out.

The efficacy of monovalent Fab fragments of specific mAbs in normalizing human colorectal carcinoma cells in vivo was assessed by a treatment protocol designed in which the ability of the treated cells to conform to normal colonic tissue architecture and to fail to form tumors was applied. The treatment concept is to apply a composition comprising the Fab fragment of A20, which should decluster CEA/CEACAM6 molecules on the tumor cell surfaces, thus forcing them to resume cell polarity, differentiation, normal tissue architecture and restored anoikis.

Several assays have been carried out in which control treatment was compared with A-20 Fabs containing composition on two human colorectal carcinoma cell lines (test cells) mixed in a defined proportion with normal fetal rat colonic cells and grown as aggregates implanted under the kidney capsule of nude mice. The test cells in the “mini-colons” that develop after 7-10 days were then assessed for either conformance to normal crypt-like tissue architecture or for formation of tumors (et al. 43).

The primary limitation of most antibody fragments is high renal uptake and subsequent catabolism which consequently limits imaging and therapeutic applications. With this in mind, the treatment regimen employed comprised a subjecting of the test cells to the maximum possible dose over the course of the assay. This regimen includes three administrations of A-20 Fabs to the test cells in the ex vivo portion of the assay during which the aggregates are constructed (pre-treatment), followed by daily intravenous injections during the in vivo growth of the implanted aggregates (therapeutic treatment). Pre-treatment of the test cells with A-20 Fabs at 100 μg/ml commenced on day 0. Cells were washed to remove excess unbound Fab, mixed with dissociated fetal rat colonic tissue, and shaken on a rotary platform with 100 μg/ml A-20 Fab present in the medium. Fresh Fab was added the following day (cells were in aggregate form at this point). Aggregates were implanted on day 2 followed by daily administrations of A-20 Fab [0.5 mg/dose (iv)] starting on day 3 until day 8. Mice were sacrificed and the mini-colon bearing kidney removed for analysis on day 9.

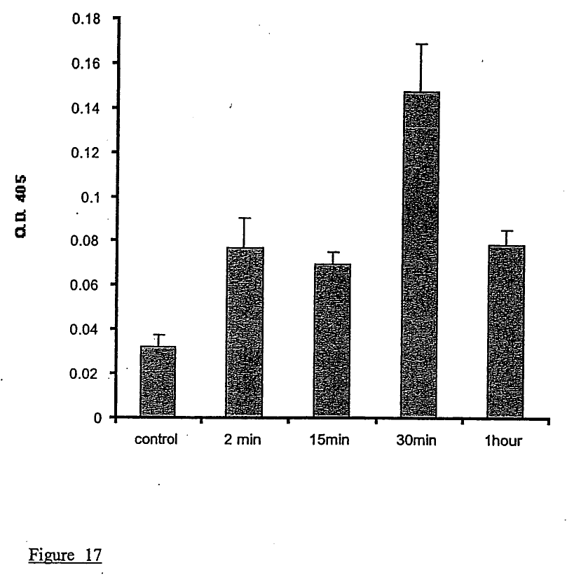

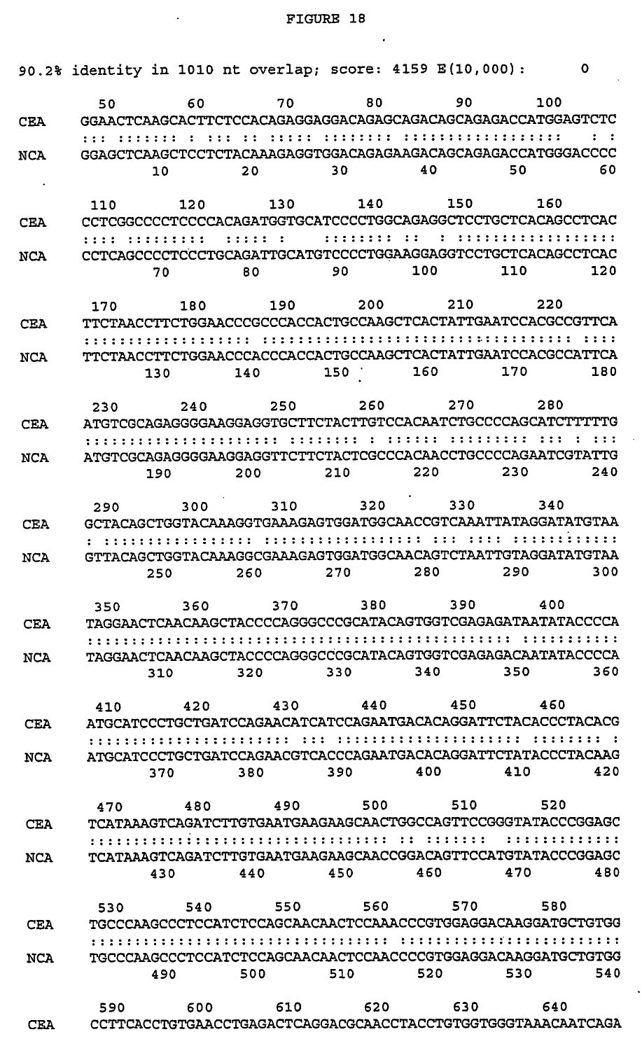

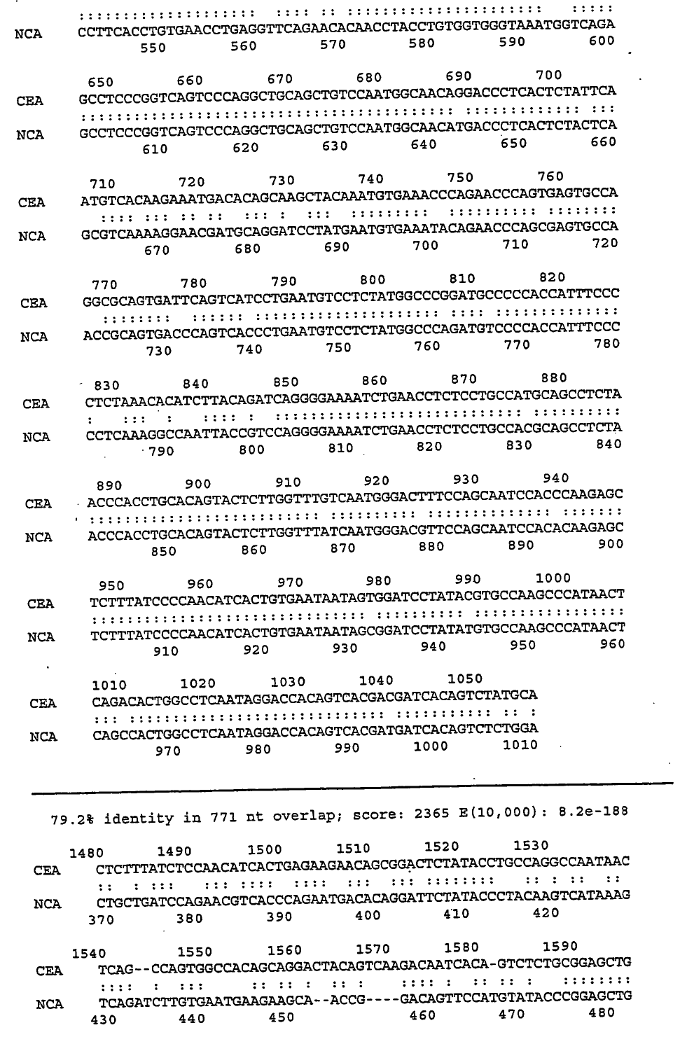

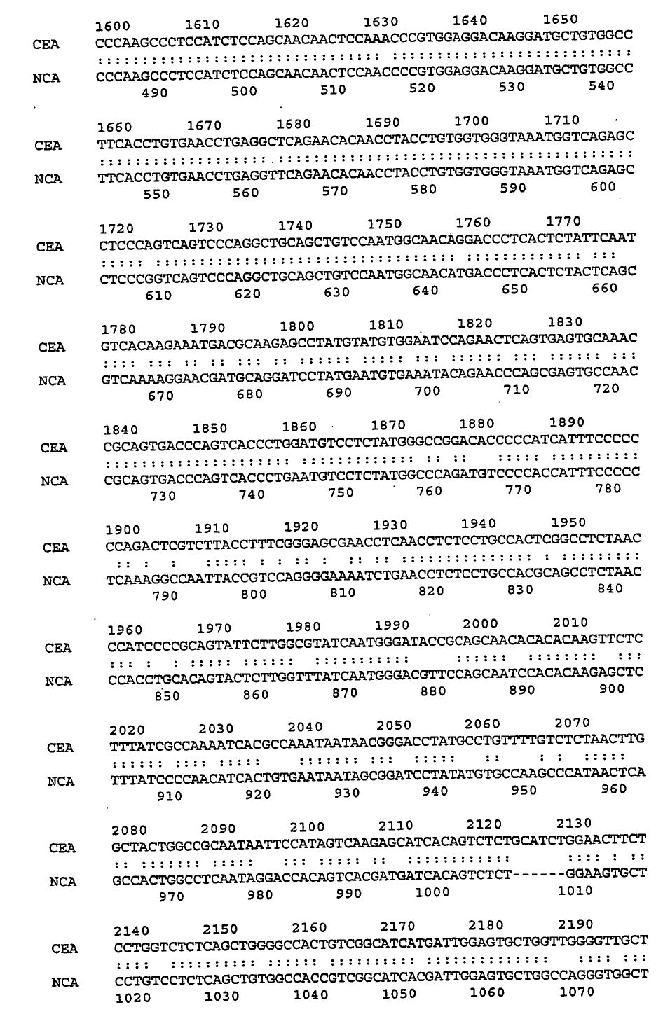

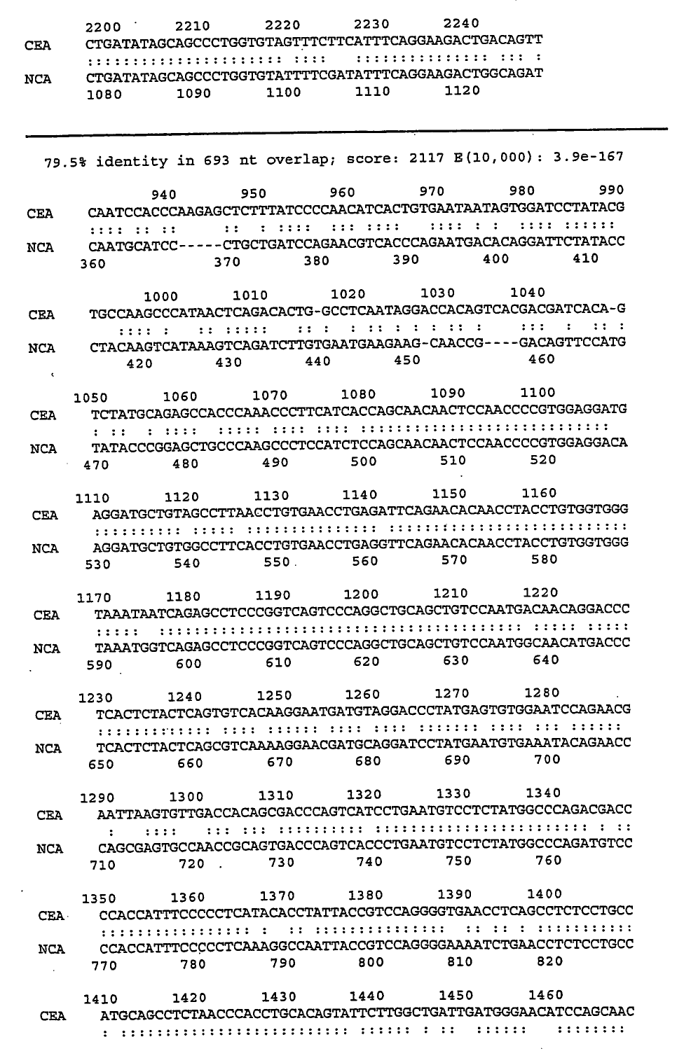

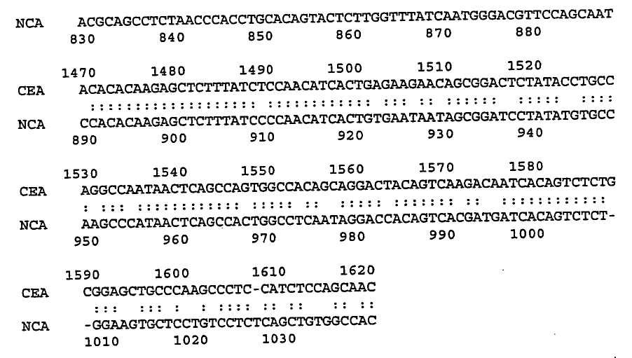

The results have revealed a significant reduction in tumor growth in animals treated with Fab versus control treatment (subjecting animal to the same composition but lacking A20 Fabs). The A-20 Fab treated animals showed dramatically reduced tumor growth (FIG. 16) and resumption of quasi-normal tissue architecture with the production of normal colonic crypts consisting of polarized differentiated cells (FIG. 17), when compared to control treated animals; the tumors in the latter were much larger, more solid and less differentiated. Results of this nature were obtained for two different human colorectal carcinoma cell lines, SW-1222 and LS-180. Control experiments have shown that Fab fragments of an irrelevant mouse mAb were ineffective in inducing such normalization.

Thus, as demonstrated herein monovalent CEA-binding agents such as CEA mAb Fab fragments can be administered to patients with cancers over-expressing CEA/CEACAM6 and will render them more normal. In the case of tumor cells still not conforming to more normal tissue architecture it should be assessed whether CEA monovalent binding agents should make them more sensitive to chemotherapeutic drug treatment.

Although the present invention has been described herein above by way of preferred embodiments thereof, it can be modified without departing from the spirit and nature of the subject invention as defined in the appended claims.

### REFERENCES

1. Ruoslahti, E., and Obrink, B. (1996) Exp Cell Res 227(1), 1-11.
2. Hynes, R. O., and Lander, A. D. (1992) Cell 68(2), 303-22.
3. Edelman, G. M., and Crossin, K. L. (1991) Annu Rev Biochem 60, 155-90.
4. Giancotti, F. G., and Ruoslahti, E. (1999) Science 285(5430), 1028-32.
5. Obrink, B. (1997) Curr Opin Cell Biol 9(5), 616-26.
6. Huang, Z., Li, S., and Korngold, R. (1998), pp. 367-382, John Wiley and Sons, Inc.
7. Williams, A. F., and Barclay, A. N. (1988) Annu Rev Immunol 6, 381-405.
8. Amzel, L. M., and Poljak, R. J. (1979) Annu Rev Biochem 48,961-97.
9. Zhou, H., Fuks, A., Alcaraz, G., Bolling, T. J., and Stanners, C. P. (1993) J Cell Biol 122(4), 951-60.
10. Oikawa, S., Inuzuka, C., Kuroki, M., Arakawa, F., Matsuoka, Y., Kosaki, G., and Nakazato, H. (1991) J Biol Chem 266(13), 7995-8001.
11. Ranheim, T. S., Edelman, G. M., and Cunningham, B. A. (1996) Proc Natl Acad Sci USA 93(9), 4071-5.
12. Knudson, A. G., Jr. (1995) Adv Cancer Res 67, 1-23.
13. Stanners, C. P., and Fuks, A. (1998) in Cell Adhesion and Communication Mediated by the CEA Family: Basic and Clinical Perspectives (Stanners, C. P., Ed.), pp. 57-71, Harwood academic publishers, Amsterdam.
14. Brieher, W. M., Yap, A. S., and Gumbiner, B. M. (1996) J Cell Biol 135(2), 487-96.
15. Draber, P., and Skubitz, K. M. (1998) in Cell Adhesion and Communication Mediated by the CEA Family: Basic and Clinical Perspectives (Stanners, C. P., Ed.), pp. 121-140, Harwood academic publishers, Amsterdam.
16. Taheri, M., Saragovi, U., Fuks, A., Makkerh, J., Mort, J., and Stanners, C. P. (2000) Journal of Biological Chemistry 275(35), 26935-26943.
17. Beauchemin, N., Benchimol, S., Cournoyer, D., Fuks, A., and Stanners, C. P. (1987) Mol Cell Biol 7(9), 3221-30.
18. Hefta, S. A., Hefta, L. J., Lee, T. D., Paxton, R. J., and Shively, J. E. (1988) Proc Natl Acad Sci USA 85(13), 4648-52.
19. Takami, N., Misumi, Y., Kuroki, M., Matsuoka, Y., and Ikehara, Y. (1988) J Biol Chem 263(25), 12716-20.
20. Eidelman, F. J., Fuks, A., DeMarte, L., Taheri, M., and Stanners, C. P. (1993) J Cell Biol 123(2), 467-75.
21. Screaton, R. A., Penn, L. Z., and Stanners, C. P. (1997) J Cell Biol 137(4), 939-52.
22. Ilantzis, C., Jothy, S., Alpert, L. C., Draber, P., and Stanners, C. P. (1997) Lab Invest 76(5), 703-16.
23. Ordonez, C., Screaton, R. A., llantzis, C., and Stanners, C. P. (2000) Cancer Research 60(13), 3419-24.
24. Chevinsky, A. H. (1991) Semin Surg Oncol 7(3), 162-6.
25. Higuchi, R. (1989) Using PCR to Engineer DNA. PCR Technology: Principles and Applications for DNA Amplification (Erlich, H. A., Ed.), cold Spring Harbor laboratory Press.
26. Pollard, J. W., and Stanners, C. P. (1979) J Cell Physiol 98(3), 571-85.
27. Stanners, C. P., Eliceiri, G. L., and Green, H. (1971) Nat New Biol 230(10), 52-4.
28. Benchimol, S., Fuks, A., Jothy, S., Beauchemin, N., Shirota, K., and Stanners, C. P. (1989) Cell 57(2), 327-34.
29. Zhou, H., Stanners, C. P., and Fuks, A. (1993) Cancer Res 53(16), 3817-22.
30. De Giovanni, C., Lollini, P. L., Dolccetti, R., Landuzzi, L., Nicoletti, G., D'Andrea, E., Scotland, K., and Nanni, P. (1993) British Journal of Cancer 67, 674-679.
31. Sippel, C. J., Shen, T., and Perlmutter, D. H. (1996) J. Biol. Chem. 271(51), 33095-104.
32. Stanners, C. P. (1998) in Cell Adhesion and Communication Mediated by the CEA Family: Basic and Clinical Perspectives (Stanners, C. P., Ed.), pp. 141-154, Harwood academic publishers, Amsterdam.
33. Sastry, S. K., Lakonishok, M., Thomas, D. A., Muschler, J., and Horwitz, A. F. (1996) J Cell Biol 133(1), 169-84.
34. Gullberg, D., Sjoberg, G., Velling, T., and Sejersen, T. (1995) Exp Cell Res 220(1), 112-23.
35. Boettiger, D., Enomoto-lwamoto, M., Yoon, H. Y., Hofer, U., Menko, A. S., and Chiquet-Ehrismann, R. (1995) Dev Biol 169(1), 261-72.
36. von der Mark, K., and Ocalan, M. (1989) Differentiation 40(2), 150-7.
37. Hauck, W., and Stanners, C. P. (1991) Cancer Res 51(13), 3526-33.
38. Yamanaka, T., Kuroki, M., Matsuo, Y., and Matsuoka, Y. (1996) Biochem Biophys Res Commun 219(3), 842-7.
39. Oikawa, S., Inuzuka, C., Kuroki, M., Matsuoka, Y., Kosaki, G., and Nakazato, H. (1989) Biochem Biophys Res Commun 164(1), 39-45.
40. Screaton, R. A., DeMarte, L., Draber, P., and Stanners, C. P., (2000) J. Cell Biol 150, 613-26.
41. Ilantzis, C., DeMarte, L., Screaton, R. A., and Stanners, C. P. (2002) Neoplasia, 4(2), 151-63.
42. Taheri et al., 2003 J. Cell Biol. 278, 14632-14639.
43. llantzis, C., et al. 1997, In Vitro Cell. Dev. Biol. Animal 33, 50-61.

What is claimed is:

1. A monovalent CEA binding agent that inhibits intracellular or intercellular CEA-CEA interactions responsible for CEA-mediated tumorigenic effects.

2. The agent of claim 1, wherein the agent causes at least a partial reversal of the CEA-mediated differentiation block.

3. The agent of claim 1 wherein the agent inhibits CEA-CEA interactions involving N-terminal domains responsible for CEA-mediated tumorigenic effects.

4. The agent of claim 1 wherein the agent inhibits CEA-CEA interactions that lead to activation of integrins.

5. The agent of claim 4 wherein the agent inhibits activation of integrin α₅β₁.

6. The agent of claim 1 that also inhibits intercellular adhesion promoted by CEA.

7. The agent of claim 1 wherein the agent is a peptide.

8. The agent of claim 7 wherein the peptide is selected from the group consisting of a synthetic peptide, a cyclic peptide and a fragment of an antibody.

9. The agent of claim 8 selected from the group consisting of cyclized H-CGYSWYKC-OH, H-CGNRQIIC-OH and H-CQNDTGC-OH and a Fab fragment or monovalent ScFv fragment of an anti-CEA antibody.

10. The agent of claim 9 wherein the Fab fragment or ScFv fragment interacts with an epitope in the N-terminal region of CEA.

11. The agent of claim 10, wherein the agent is a monovalent Fab fragment derived from the A-20 monoclonal antibody.

12. The agent of claim 11 wherein the agent interacts with an epitope of CEA that includes amino acids K₃₅ and N₄₂ of the N-terminal region of CEA.

13. The agent of claim 1, wherein the agent is a humanized antibody fragment.

14. A method to identify monovalent CEA declustering agents which can interfere with the CEA interactions responsible for CEA-mediated tumorigenic effects, comprising performing a biological assay in the presence of a candidate monovalent declustering agent to measure differentiation or detect a reduction of tumorigenic effects, and selecting said candidate monovalent declustering agent when said differentiation block, or marker thereof, or said tumorigenic effect, is minimized, reduced or ablated in the presence of said candidate agent as compared to in the absence thereof.

15. A method of reducing or preventing a CEA-mediated tumorigenic effect in a cell or tissue, comprising contacting said cell or tissue with the agent of claim 1.

16. A method of reducing or preventing a CEA-mediated tumorigenic effect in a cell or tissue, comprising contacting said cell or tissue with the agent of claim 11.

17. A method of reducing or preventing a CEA-mediated tumorigenic effect in a cell or tissue, comprising contacting said cell or tissue with the agent of claim 12.

18. The method of claim 15 wherein the agent reduces or prevents the tumorigenic effects by inhibiting N-terminal domain interactions of CEA.

19. The method of claim 15 wherein the agent is a peptide.

20. The method of claim 19 wherein the peptide is selected from the group consisting of a synthetic peptide, a cyclic peptide or a monovalent fragment of an antibody.

21. The method of claim 20 wherein the cyclic peptide is selected from the group consisting of cyclic peptides having the sequence
H-CGYSWYKC-OH, H-CGNRQIIC-OH AND H-CQNDTGC-OH.

22. The method of claim 20 wherein the monovalent fragment of an antibody is selected from the group consisting of a Fab fragment or ScFv of an anti-CEA antibody.

23. The method of claim 20 wherein the Fab fragment or ScFv fragment interacts with an epitope in the N-terminal domain of CEA.

24. The method of claim 15 wherein the agent binds to an N-terminal region of CEA having an amino acid sequence selected from the group of sequences consisting of GYSWYK, NRQII and QNDTG.

25. The method of claim 15 wherein the agent is a humanized antibody fragment.

26. A pharmaceutical composition comprising the agent of claim 1 in combination with a pharmaceutically acceptable carrier.

27. The pharmaceutical composition of claim 26 wherein the agent is an antibody that interacts with an N-terminal region of CEA having an amino acid sequence selected from the group of sequences consisting of GYSWYK, NRQII and QNDTG.

28. The pharmaceutical composition of claim 26 wherein the agent is a Fab fragment of the A20 antibody.


SEQUENCE LISTING (15 sequences)

SEQ ID NO: 1 - DNA, 3036 bp, Homo sapiens
  CDS (97)..(2202)
cgaccagcagaccagacagtcacagcagccttgacaaaacgttcctggaactcaagcacttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccatgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattctttaaatgtcttgtttcccagatttcaggaaactttttttcttttaagctatccacagcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtatgacagaatacatttgaaaacattggttatattaccaagactttgactagaatgtcgtat

SEQ ID NO: 2 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 3 - DNA, 2533 bp, Homo sapiens
  CDS (51)..(1082)
ggagctcaagctcctctacaaagaggtggacagagaagacagcagagaccatgggacccccctcagcccctccctgcagattgcatgtcccctggaaggaggtcctgctcacagcctcacttctaaccttctggaacccacccaccactgccaagctcactattgaatccacgccattcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggaagtgctcctgtcctctcagctgtggccaccgtcggcatcacgattggagtgctggccagggtggctctgatatagcagccctggtgtattttcgatatttcaggaagactggcagattggaccagaccctgaattcttctagctcctccaatcccattttatcccatggaaccactaaaaacaaggtctgctctgctcctgaagccctatatgctggagatggacaactcaatgaaaatttaaagggaaaaccctcaggcctgaggtgtgtgccactcagagacttcacctaactagagacagtcaaactgcaaaccatggtgagaaattgacgacttcacactatggacagcttttcccaagatgtcaaaacaagactcctcatcatgataaggctcttacccccttttaatttgtccttgcttatgcctgcctctttcgcttggcaggatgatgctgtcattagtatttcacaagaagtagcttcagagggtaacttaacagagtgtcagatctatcttgtcaatcccaacgttttacataaaataagagatcctttagtgcacccagtgactgacattagcagcatctttaacacagccgtgtgttcaaatgtacagtggtccttttcagagttggacttctagactcacctgttctcactccctgttttaattcaacccagccatgcaatgccaaataatagaattgctccctaccagctgaacagggaggagtctgtgcagtttctgacacttgttgttgaacatggctaaatacaatgggtatcgctgagactaagttgtagaaattaacaaatgtgctgcttggttaaaatggctacactcatctgactcattctttattctattttagttggtttgtatcttgcctaaggtgcgtagtccaactcttggtattaccctcctaatagtcatactagtagtcatactccctggtgtagtgtattctctaaaagctttaaatgtctgcatgcagccagccatcaaatagtgaatggtctctctttggctggaattacaaaactcagagaaatgtgtcatcaggagaacatcataacccatgaaggataaaagccccaaatggtggtaactgataatagcactaatgctttaagatttggtcacactctcacctaggtgagcgcattgagccagtggtgctaaatgctacatactccaactgaaatgttaaggaagaagatagatccaattaaaaaaaattaaaaccaatttaaaaaaaaaaaagaacacaggagattccagtctacttgagttagcataatacagaagtcccctctactttaacttttacaaaaaagtaacctgaactaatctgatgttaaccaatgtatttatttctgtggttctgtttccttgttccaatttgacaaaacccactgttcttgtattgtattgcccagggggagctatcactgtacttgtagagtggtgctgctttaattcataaatcacaaataaaagccaattagctctataact

SEQ ID NO: 4 - protein, 344 aa, Homo sapiens
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 5 - protein, 6 aa, Homo Sapiens
GYSWYK

SEQ ID NO: 6 - protein, 5 aa, Homo Sapiens
NRQII

SEQ ID NO: 7 - protein, 5 aa, Homo Sapiens
QNDTG

SEQ ID NO: 8 - protein, 10 aa, Artificial Sequence
  Synthetic blocked linear peptide
LFGYSWYKGE

SEQ ID NO: 9 - protein, 10 aa, Artificial Sequence
  Synthetic blocked linear peptide
VDGNRQIIGY

SEQ ID NO: 10 - protein, 10 aa, Artificial Sequence
  Synthetic blocked linear peptide
RIIQNDTGFY

SEQ ID NO: 11 - protein, 9 aa, Artificial Sequence
  Synthetic blocked linear peptide
FNVAEGKEV

SEQ ID NO: 12 - protein, 8 aa, Artificial Sequence
  Synthetic cyclic peptide
CGYSWYKC

SEQ ID NO: 13 - protein, 8 aa, Artificial Sequence
  Synthetic cyclic peptide
CGNRQIIC

SEQ ID NO: 14 - protein, 7 aa, Artificial Sequence
  Synthetic cyclic peptide
CQNDTGC

SEQ ID NO: 15 - protein, 9 aa, Artificial Sequence
  Synthetic cyclic peptide
YCTDEKQCY